SEQUENCE LISTING (12 sequences)

SEQ ID NO: 1 - protein, 585 aa, Artificial Sequence
  Human heptocytes cell lines were treated with rHA or His67ALa mutant albumin to study their effects on the human heptocyte cell culture. The cell line used was WRL-68.
  misc_feature (30)..(30): X IS ANY AMINO ACID OTHER THAN Y
  misc_feature (67)..(67): X IS ANY AMINO ACID OTHER THAN H
  misc_feature (99)..(100): X IS ANY AMINO ACID OTHER THAN N
  misc_feature (103)..(103): X IS ANY AMINO ACID OTHER THAN E
  misc_feature (146)..(146): X IS ANY AMINO ACID OTHER THAN L
  misc_feature (242)..(242): X IS ANY AMINO ACID OTHER THAN H
  misc_feature (247)..(247): X IS ANY AMINO ACID OTHER THAN H
  misc_feature (248)..(248): X IS ANY AMINO ACID OTHER THAN G
  misc_feature (249)..(249): X IS ANY AMINO ACID OTHER THAN D
  misc_feature (288)..(288): X IS ANY AMINO ACID OTHER THAN H
DAHKSEVAHRFKDLGEENFKALVLIAFAQXLQQCPFEDHVKLVNEVTEFAKTCVADESAENCDKSLXTLFGDKLCTVATLRETYGEMADCCAKQEPERXXCFXQHKDDNPNLPRLVRPEVDVMCTAFHDNEETFLKKYLYEIARRXPYFYAPELLFFAKRYKAAFTECCQAADKAACLLPKLDELRDEGKASSAKQRLKCASLQKFGERAFKAWAVARLSQRFPKAEFAEVSKLVTDLTKVXTECCXXXLLECADDRADLAKYICENQDSISSKLKECCEKPLLEKSXCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 609 aa, Artificial [sequence omitted]

SEQ ID NO: 3 - protein, 600 aa, Rhesus macaque
LLFLFSSAYSRGVFRRDTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKFVAASQAALA

SEQ ID NO: 4 - protein, 608 aa, Canis familiaris
DRNECFLAHKDDNPGFPPLVAPEPDALCAAFQDNEQLFLGKYLYEIARRHPYFYAPELLYEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYSRRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCADDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 5 - protein, 608 aa, Felis catus
MKWVTFISLLLLFSSAYSRGVTRFSAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 6 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 7 - protein, 607 aa, Ovis aries
MKWVTFISLLLLFSSAYSRGVFRRDTHKSEIAHRFNDLGEENFQGLVLIAFSQYLQQCPFDEHVKLVKELTEFAKTCVADESHAGCDKSLHTLFGDELCKVATLRETYGDMADCCEKQEPERNECFLNHKDDSPDLPKLKPEPDTLCAEFKADEKKFWGKYLYEVARRHPYFYAPELLYYANKYNGVFQECCQAEDKGACLLPKIDAMREKVLASSARQRLRCASIQKFGERALKAWSVARLSQKFPKADFTDVTKIVTDLTKVHKECCHGDLLECADDRADLAKYICDHQDALSSKLKECCDKPVLEKSHCIAEVDKDAVPENLPPLTADFAEDKEVCKNYQEAKDVFLGSFLYEYSRRHPEYAVSVLLRLAKEYEATLEDCCAKEDPHACYATVFDKLKHLVDEPQNLIKKNCELFEKHGEYGFQNALIVRYTRKAPQVSTPTLVEISRSLGKVGTKCCAKPESERMPCTEDYLSLILNRLCVLHEKTPVSEKVTKCCTESLVNRRPCFSDLTLDETYVPKPFDEKFFTFHADICTLPDTEKQIKKQTALVELLKHKPKATDEQLKTVMENFVAFVDKCCAADDKEGCFVLEGPKLVASTQAALA

SEQ ID NO: 8 - protein, 605 aa, Sus scrofa
WVTFISLLFLFSSAYSRGVFRRDTYKSEIAHRFKDLGEQYFKGLVLIAFSQHLQQCPYEEHVKLVREVTEFAKTCVADESAENCDKSIHTLFGDKLCAIPSLREHYGDLADCCEKEEPERNECFLQHKNDNPDIPKLKPDPVALCADFQEDEQKFWGKYLYEIARRHPYFYAPELLYYAIIYKDVFSECCQAADKAACLLPKIEHLREKVLTSAAKQRLKCASIQKFGERAFKAWSLARLSQRFPKADFTEISKIVTDLAKVHKECCHGDLLECADDRADLAKYICENQDTISTKLKECCDKPLLEKSHCIAEAKRDELPADLNPLEHDFVEDKEVCKNYKEAKDVFLGTFLYEYSRRHPDYSVSLLLRIAKIYEATLEDCCAKEDPPACYATVFDKFQPLVDEPKNLIKQNCELFEKLGEYGFQNALIVRYTKKVPQVSTPTLVEVARKLGLVGSRCCKRPEEERLSCAEDYLSLVLNRLCVLHEKTPVSEKVTKCCTESLVNRRPCFSALTPDETYKPKEFVEGTFTFHADLCTLPEDEKQIKKQTALVELLKHKPHATEEQLRTVLGNFAAFVQKCCAAPDHEACFAVEGPKFVIEIRGILA

SEQ ID NO: 9 - protein, 608 aa, Oryctolagus cuniculus
MKWVTFISLLFLFSSAYSRGVFRESAHKSEIAHRFNDVGEEHFIGLVLITFSQYLQKCPYEEHAKLVKEVTDLAKACVADESAANCDKSLHDIFGDKICALPSLRDTYGDVADCCEKKEPERNECFLHHKDDKPDLPPFARPEADVLCKAFHDDEKAFFGHYLYEVARRHPYFYAPELLYYAQKYKAILTECCEAADKGACLTPKLDALEGKSLISAAQERLRCASIQKFGDRAYKAWALVRLSQRFPKADFTDISKIVTDLTKVHKECCHGDLLECADDRADLAKYMCEHQETISSHLKECCDKPILEKAHCIYGLHNDEDTAGLPAVAEEFVEDKDVCKNYEEAKDLFLGKFLYEYSRRHPDYSVVLLLRLGKAYEATLKKCCATDDPHACYAKVLDEFQPLVDEPKNLVKQNCELYEQLGDYNFQNALLVRYTKKVPQVSTPTLVEISRSLGKVGSKCCKHPEAERLPCVEDYLSVVLNRLCVLHEKTPVSEKVTKCCSESLSNRRPCFSALGPDETYVPKEFNAETFTFHADICTLPETERKIKKQTALVELVKHKPHATNDQLKTVVGEFTALLDKCCSAEDKEACFAVEGPKLVESSKATLG

SEQ ID NO: 10 - protein, 608 aa, Rattus norvegicus
MKWVTFLLLLFISGSAFSRGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKANAVARMSQRFPNAEFAEITKLATDVTKINKECCHGDLLECADDRAELAKYMCENQATISSKLQACCDKPVLQKSQCLAETEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 11 - DNA, 50 bp, Artificial Sequence
  Oligonucleotide-directed mutagenesis was used to prepare cDNAs encoding the H67A mutated form of albumin. The mutagenic oglinucleotide, supplied by Delta Biotechnology Ltd. in Nottingham, Massachusetts, that was used is listed below.
gctgaaattgtgacaaatcacttgctaccctttttggagacaaattatgc

SEQ ID NO: 12 - DNA, 51 bp, Artificial Sequence
  Oligonucleotide-directed mutagenesis was used to prepare cDNAs encoding the H67A mutated form of albumin. The mutagenic oglinucleotide, supplied by Delta Biotechnology Ltd. in Nottingham, Massachusetts, that was used is listed below.
gcataatttgtctccaaaaagggtagcaagtgatttgtcacaattttcagc

(19) United States
(12) Patent Application Publication
(10) Pub. No.: US 2007/0185315 A1
(43) Pub. Date: Aug. 9, 2007

(54) Novel albumins

(76) Inventors: Stephen Berezenko, Loughborough (GB); Peter John Sadler, Penicuik (GB); Alan James Stewart, Edinburgh (GB); Claudia Blindauer, Coventry (GB); Kerry Emma Bunyan, Edinburgh (GB)

Correspondence Address: MORGAN, LEWIS & BOCKIUS, LLP., 2 PALO ALTO SQUARE, 3000 EL CAMINO REAL, PALO ALTO, CA, 94306, US

(21) Appl. No.: 10/523,312

(22) PCT Filed: Jul. 28, 2003
(86) PCT No.: PCT/GB03/03199

§ 371 Date: Oct. 25, 2005

(30) Foreign Application Priority Data
Jul. 26, 2002 (GB) 0217347.4

(51) Int. Cl. A61K 38/38; C07H 21/04; C12P 21/06; C07K 14/765

(52) U.S. Cl. 530/363; 514/12; 536/23.5; 435/69.1; 435/320.1; 435/325

(57) ABSTRACT

The present invention relates to mutated forms of serum albumin, which display altered metal binding and/or other characteristics with respect to a native albumin from which the mutant has been derived, as well as uses of such mutant albumins in the medical field or in growth of cells in culture.

### FIELD OF THE INVENTION

The present invention relates to mutated forms of serum albumin, which display altered metal binding and/or other characteristics with respect to a native albumin from which the mutant has been derived, as well as uses of such mutant albumins in the medical field or in growth of cells in culture.

### BACKGROUND OF THE INVENTION

Human albumin is the most abundant protein in blood plasma. Typically, it is present at concentrations of around 750 μM. It is a single polypeptide chain of 585 amino acids with a largely helical triple-domain structure. The gene for human serum albumin comprises 16,961 nucleotides from the supposed “capping” site up to the first site for addition of poly(A).

Albumin is the major transport protein in the blood and can reversibly bind to a wide range of small molecules, such as fatty acids, hormones, and drugs. Albumin is also implicated in the transport and storage of many metal ions. Presently, human albumin is used clinically in the treatment of patients with severe burns, shock or blood loss. Other mammalian albumins are highly homologous with human albumin.

Zinc and copper are known to bind albumin with association constants of 3.4×10⁷ and 1.5×10×16 M⁻¹ respectively (Masuoka et al. (1993) J. Biol. Chem. 268, 21533-21537). Cu²⁺ binds most strongly to albumin's N-terminal amino acids Asp1-Ala2-His3, which provide a square-planar site of 4 N ligands, although other binding sites on the molecule are known to exist.

Approximately 75% of Zn²⁺ in blood plasma (ca. 14 μM) is bound to albumin. This accounts for as much as 98% of the exchangeable fraction of Zn²⁺ in serum (Giroux et al. (1976) J. Bioinorg. Chem. 5, 211-218; Foote and Delves (1984) Analyst 109, 709-711). Albumin has previously been shown to modulate zinc uptake by endothelial cells, whilst receptor-mediated vesicular co-transport across the endothelium has been demonstrated with albumin-zinc complexes in vitro (Bobilya et al. (1993) Proc. Soc. Exp. Biol. Med. 202, 159-166; Tibaduiza et al. (1996) J. Cell. Physiol. 167, 539-547). No binding sites for Zn²⁺ on albumin had previously been specifically located, even though albumin is believed to be the main zinc transport protein in the circulation.

Zinc is an essential element in the body and is present in over 300 enzymes. It has many important roles including the transport of vitamin A, the healing of wounds, sperm production in men and is recruited by anthrax lethal factor and bacterial enterotoxin. The regulation of zinc levels in the blood is therefore physiologically very important. It has been proposed that Zn²⁺ recruitment from blood can be used to increase the affinity of certain metal-binding organic drugs for proteins and enzymes, e.g. benzimidazole inhibitors of serine proteases such as trypsin (Katz and Luong (1999) J. Mol. Biol. 292, 669-684; Janc et al. (2000) Biochemistry 39, 4792-4800; Katz et al. (2001) Chem. & Biol. 8, 1107-1121; Liang et al. (2002) J. Am. Chem. Soc.).

Reed & Burrington (J. Biol. Chem. (1989) 264, 17, p 9867-9872) is concerned with the binding of albumin to hepatocytes and whether or not this involves a cell surface receptor for albumin. The authors propose that their work provides evidence for reversible adsorption of albumin to hepatocyte surfaces and this as accompanied by a conformational change that enhances the interaction between albumin and the hepatocyte surface. However, there is no suggestion as to what conformational changes may be occurring or how this would be controlled.

Bos et al (J. Biol. Chem (1989), 264, 2, p 953-959) is concerned with the molecular mechanism of the neutral-to-base transition of human serum albumin by binding of Ca²⁺ ions through histidine residues. The paper discloses that the N-B transition may play a role in the pharmacokinetics of drugs, but does not suggest creating mutant serum albumins or propose any effects such mutants may possess.

### SUMMARY OF THE INVENTION

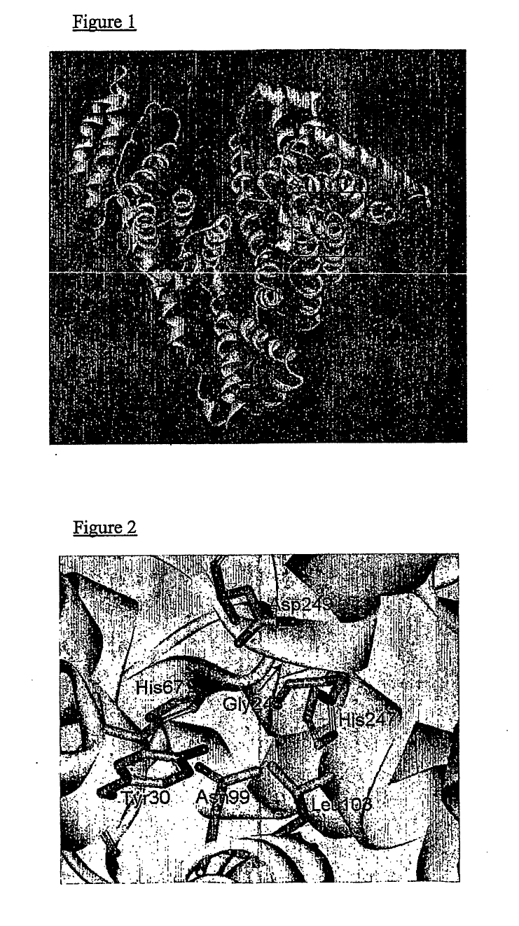

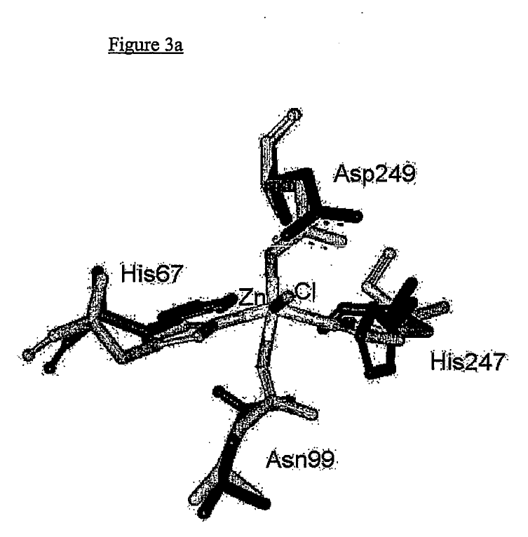

The present invention is based on the initial discovery that a cluster of four amino acids (His67, Asn99, His247 and Asp249), which lie at the interface between domains I and II are involved in a binding site for zinc, copper and/or cadmium (see FIGS. 1 and 2). All four of these residues are highly conserved amongst all mammalian albumins sequenced to date (see Table 1). The numbering referred to herein relates to the amino acid found at the particular position of the human serum albumin amino acid sequence after the prepro-albumin sequence has been cleaved following translation (see Table 1). Identification of this site provides a rationale for the design of therapeutic albumins for controlling the levels of available zinc and/or other metal ions in blood and their delivery to target tissues. The present invention is also based on observations of effects mutant albumins have on cell adhesion.

Thus, in a first aspect there is provided an isolated mutant serum albumin which has been mutated such that the mutant displays an altered metal binding affinity and/or physiological other characteristic(s) with respect to a native albumin from which the mutant has been derived.

Any mutation to a native albumin sequence which results in altered metal binding and/or other physiological characteristic(s), as hereinafter defined is envisaged to be encompassed by the present invention. It is a relatively straightforward task for the skilled addressee to generate a particular mutant and to test whether or not such a mutant displays altered metal binding and/or other physiological characteristic(s), based on the experimental tests described herein.

Preferred residues which may be mutated are identified as residues X₁-X₁₁, as identified in Table 1 and/or residues which may hydrogen bond with any of such residues which may be determined from the crystal structure determined for a particular serum albumin.

In a second aspect there is provided an isolated mutant human serum albumin substantially comprising the amino acid sequence:

DAHKSEVAHRFKDLGEENFKALVLIAFAQX₅LQQCPFEDHVKLVNEVTEF
AKTCVADESAENCDKSLX₁TLFGDKLCTVATLRETYGEMADCCAKQEPER
X₂X₈CFX₆QHKDDNPNLPRLVRPEVDVMCTAFHDNEETFLKKYLYEIARR
X₉PYFYAPELLFFAKRYKAAFTECCQAADKAACLLPKLDELRDEGKASSA
KQRLKCASLQKFGERAEKAWAVARLSQRFPKAEFAEVSKLVTDLTKVX₁₀
TECCX₃X₇X₄LLECADDRADLAKYICENQDSISSKLKECCEKPLLEKS
X₁₁CIAEVENDEMPADLPSLAADFVESKDVCKNYAEAKDVFLGMFLYEYA
RRHPDYSVVLLLRLAKTYETTLEKCCAAADPHECYAKVFDEFKPLVEEPQ
NLIKQNCELFEQLGEYKFQNALLVRYTKKVPQVSTPTLVEVSRNLGKVGS
KCCKHPEAKRMPCAEDYLSVVLNQLCVLHEKTPVSDRVTKCCTESLVNRR
PCFSALEVDETYVPKEFNAETFTFHADICTLSEKERQIKKQTALVELVKH
KPKATKEQLKAVMDDFAAFVEKCCKADDKETCFAEEGKKLVAASQAALGL

wherein X₁, is other than H; X₂ is other than N, X₃ is other than H, X₄ is other than D; X₅ is other than Y; X₆ is other than L; X₇ is other than G, X₈ is other than E, X₉ is other than H, X₁₀ is other than H, and/or X₁₁ is other than H, such that said mutant displays an altered metal binding affinity and/or other physiological characteristic(s) with respect to native human serum albumin.

It is to be understood that conventional one-letter amino acid nomenclature is used throughout. Amino acid substitutions may be for other natural amino acids, especially the 20 amino acids that are encoded directly by DNA, or could for example be synthetic or unusual amino acids known to those skilled in the art. The sequence shown above is based on the sequence of human serum albumin found in the Genbank database. Human serum albumin comprises the sequence identified above, wherein X₁ is H, X₂ is N, X₃ is H, X₄ is D, X₅ is Y, X₆ is L, X₇ is G, X₈ is E, X₉ is H, X₁₀ is H and X₁₁ is H. Thus, the mutant serum albumins according to the present invention typically comprise at least one mutation at positions X₁ to X₁₁ with respect to a natural amino acid of a particular species “albumin” found at said position. Nevertheless, it will be appreciated that natural variations can exist between individuals of a species such that minor variations in sequence can occur. Such minor variations in sequence, other than the variations identified in positions X₁-X₇ are understood not to depart from the present invention. It is to be appreciated that such variations in sequence may be manifested as substitutions, inversions, deletions or translocations. However, such variant albumin sequences should display a high degree of similarity to any of the sequences shown in FIG. 1. Typically the variant albumin sequences should display at least 90%, preferably at least 95% or even 99% identity (the X positions excepted) with an identified sequence.

Homology (i.e. identity) between amino acid sequences can be determined using commercially available algorithms. The programs BLAST, gapped BLAST, BLASTN, PSI-BLAST and BLAST 2 sequences (provided by the National Center for Biotechnology Information) are widely used in the art for this purpose, and can align homologous regions of two amino acid sequences. These may be used with default parameters to determine the degree of homology between the amino acid sequence of the protein of known structure and other target proteins which are to be modelled.

It is to be understood that the mutant serum albumin is isolated in the sense that it is free or substantially or partially free of other proteins with which it may be associated in the proteome of an organism and does not therefore encompass any native forms of albumin within the proteome of a cell or organism.

The above sequence is based on the human form of serum albumin after a leader sequence (ie. MKWVTFISLLFLFSSAYSRGVFRR) has been cleaved from the sequence. The present invention also extends to mutant sequences including such leader sequences.

While the above relates to mutants of human serum albumin, it is to be understood that the present invention is not limited to only mutant human serum albumins. Serum albumins across all species display a high degree of conservation and it is well within the expertise of the skilled addressee to identify the amino acids in the positions represented by Xs in the sequence above, from albumins of other species and change said amino acids in order to alter metal binding and/or other physiological characteristic(s). Table 1 in fact shows an alignment of mammalian serum albumin polypeptide sequences in which the residues which may be mutated according to the present invention, are highlighted. It is understood that at least one of said residues should be other than the identified native residue in order to generate a mutant serum albumin, which can display altered metal binding and/or other physiological characteristic(s) with respect to the native species serum albumin.

The sequences of many serum albumins are known and readily available from the Genbank database at, for example, the National Center for Biotechnology Information: www.ncbi.nlm.nih.gov. The human sequence may for example be found under accession number P02768. Other accession numbers may also be found at www.albumin.org.

It is understood that the mutants of the present invention may be substantially similar in terms of general overall folding with respect to the native serum albumin of the particular species. For example circular dichroism studies may be conducted to see whether or not signs and magnitudes of circular dichroism bands of a mutant serum albumin are similar to native serum albumin. If they are similar this would be indicative of the mutant serum albumin displaying similar secondary structure to the native serum albumin.

The mutants of the present invention should display an altered metal binding affinity with respect to the native albumin from which the mutant is derived or other altered characteristics e.g. cell adhesion and/or growth alteration in culture. Altered metal binding affinity is understood to mean a decrease or increase in metal binding affinity (e.g. Kd) and/or an increase or decrease in the rate of binding/dissociation of the metal. Preferably the increase or decrease by a factor of 2, 4 or 6, such as a factor of 10 or 100 when looking at Kd values in terms of tog Kd values as determined in physiological conditions (i.e. about pH7.3) and appropriate with concentrations and a temperature of about 20° C.-37° C. The metals, which may display altered binding affinity to such mutant albumins, are zinc, copper, nickel and cobalt. Preferably the mutant albumins display altered binding affinity for zinc. Generally, the altered metal binding affinity will be with respect to a metal ion, such as Zn²⁺, Cu²⁺, etc. Mutation of residues thought to be involved with metal binding to residues which do not possess appropriate metal binding side chains are postulated to result in decreased metal binding affinity. Conversely mutation of residues not involved in metal/metal ion binding, but which are in the vicinity of the residues which are thought to be involved in binding to metal, to residues which assist/facilitate binding, would be expected to increase metal binding affinity.

For example, the following mutations are postulated to result in decreased metal binding affinity:

X₁ [symbol] A, F, G, I, K, L, N, P, Q, R, S, T, V, W, Y

X₂ [symbol] A, F, G, I, K, L, P, Q, R, S, T, V, W, Y

X₃ [symbol] A, F, G, I, K, L, N, P, Q, R, S, T, V, W, Y

X₄ [symbol] A, F, G, I, K, L, N, P, Q, R, S, T, V, W, Y

and mutation of a side-chain to introduce a metal binding ligand that is likely to give rise to increased or modulated metal affinity include:

X₅ [symbol] C, D, E, H (this is already a His residue in pig albumin)

X₆ [symbol] C, D, E, H

X₇ [symbol] C, D, E, H

X₂ [symbol] C, D, E, H

X₄ [symbol] C, E, H

X₁ [symbol] C, D, E

X₃ [symbol] C, D, E

The inventors have found that metal binding at the proposed site is influenced by fatty acid binding (A. J. Stewart, C. A. Blindauer, S. Berezenko, D. Sleep, P. J. Sadler, Proc. Natl. Acad. Sci. USA 100, 3701-3706 (2003).). Comparison of the X-ray structures of fatty-acid free albumin and albumin with 5 molecules of myristate (pdb 1bj5) bound reveal that, in order to accommodate a fatty acid anion in the so-called binding site 2, the long helix connecting domains I and II bends, and the two half-sites in unliganded rHA move by more than 10 Å to form a continuous cavity (Curry, S., Mandelkow, H., Brick, P. & Franks, N. (1998) Nat. Struct. Biol. 5, 827-835.). This fatty acid binding results in a movement of residues H247 and D249 by 4-6 Å away from the other two residues, H67 and N99, in the proposed Zn site (see Figure X3e (a & b). D249 also changes its side-chain conformation to maintain the H-bond to N′ of H67 and forms an additional H bond to N99. H247, which is H-bonded to N99 in the unliganded structure, forms an H bond with E100 in the fatty acid-bound structures. The proposed switching of the zinc site in human albumin by fatty acid binding is an intriguing example of an allosteric interaction between an organic nutrient and an essential metal ion. Since the 1247-E100 H-bond is expected to stabilise the “switched” form, it is predicted that the following mutations of E100 might influence the interactive metal/fatty acid binding.

X₈ [symbol] A, C, F, G, H, I, K, L, N, P, Q, R, S, T, V, W, Y

Additionally, more recent studies have revealed that albumins with the mutations H67A, N99D, and N99H display properties dramatically different from the wild-type when used in cell culture media. Cell adhesion is impaired in both the H67A and N99H mutant. It is known that uptake by the liver of, e.g., fatty acids from albumin involves non-specific binding of albumin to the cell surface, and an induced conformational change of the albumin molecule (R. G. Reed, C. M. Burrington, J. Biol. Chem. 264, 9867-9872, 1989). The mutated residues are all involved in stabilising domain I-domain-II contacts via H bonds. The finding that a single mutation at the domain I/II interface has a severe impact on the effect of the mutated albumin on cells suggests that the following mutations, which refer to domain I/II histidine residues involved in inter-domain H bonds, can also have similar impact on cell adhesion and/or growth.

X₉ [symbol] A, D, E, F, G, I, K, L, N, P, Q, R, S, T, V, W, Y

X₁₀ [symbol] A, D, E, F, G, I, K, L, N, P, Q, R, S, T, V, W, Y

X₁₁ [symbol] A, D, E, F, G, I, K, L, N, P, Q R, S, T, V, W, Y

It should be appreciated that standard one-letter amino acid nomenclature is used throughout this description.

The nature of the Zn²⁺ binding site on albumin was indicated by ¹¹³Cd-NMR studies. Several mammalian albumins have two strong binding sites for Cd²⁺ with chemical shifts characteristic of N/O coordination (Sadler and Viles (1996) Inorg. Chem. 35, 4490-4496). For human albumin, ¹¹³Cd shifts of 24 and 114 ppm (relative to Cd(ClO₄)) are indicative of sites containing a single imidazole nitrogen and 2-3 imidazole nitrogens, respectively. Zn²⁺, Cu²⁺ and Ni²⁺ ions can displace Cd²⁺ from the latter of these sites in human albumin. The present inventors' molecular modeling based on the crystal structure of albumin (PDB 1AO6) suggested that the multi-metal binding site might involve the cluster His67, Asn99, His247 and Asp249. The present inventors established the location of this site through site-directed mutagenesis of His67 to alanine followed by metal competition studies with isotopically enriched cadmium using ¹¹¹Cd NMR. Conventionally this may be represented as H67A, which identifies the histidine at position 67 being mutated to alanine. Such representation is used elsewhere in the description.

Mutation of other residues e.g. tyrosine 30 (X₅) and Glycine 248 (X₇) is envisaged to affect zinc binding. Tyrosine 30 does not bind to the metal per se, but hydrogen bonds to residue 99 which is bound to the metal. Thus, mutation of residue 30 can affect the metal binding site. The backbone carbonyl of Gly248 hydrogen bonds to residue 99 and so mutation of this residue can affect the metal binding site.

The mutated albumins of the present invention may be synthesized de novo, but preferably they are produced by recombinant means well known to those skilled in the art. The mutated albumins can, for example, be derived from the native albumin by carrying out site-directed mutagenesis on the associated gene sequence and subsequent expression of the protein. Such techniques are well known and described for example in Sambrook et al (1989) Molecular Cloning: A Laboratory Manual, Cold Spring Harbor Laboratory, Cold Spring Harbor, N.Y.

The present invention therefore also extends to a nucleic acid sequence, which encodes a mutant serum albumin according to the present invention.

For recombinant production of the mutant albumin in a host organism, the nucleotide sequence encoding the mutant albumin protein may be inserted into an expression cassette to form a DNA construct designed for a chosen host and introduced into the host where it is recombinantly produced. The choice of specific regulatory sequences such as promoter, signal sequence, 5′ and 3′ untranslated sequences, enhancer and terminator appropriate for the chosen host is within the level of skill of the routine worker in the art. The resultant molecule, containing the individual elements linked in a proper reading frame, may be introduced into the chosen cell using techniques well known to those in the art, such as calcium phosphate precipitation, electroporation, biolistic introduction, virus introduction, etc. Suitable expression cassettes and vectors and methods for recombinant production of proteins are well known for host organisms such as E. coli (see e.g. Studier and Moffatt, J. Mol. Biol. 189: 113 (1986); Brosius, DNA 8: 759 (1989)), yeast (see e.g. Schneider and Guarente, Meth. Enzymol 194: 373 (1991) and insect cells (see e.g. Luckow and Summers, Bio/Technol. 6: 47 (1988) and mammalian cell (tissue culture or gene therapy) by transfection (Schenborn E T, Goiffon V. Methods Mol. Bio. 2000; 130: 135-45, Schenborn E T, Oler J. Methods Mol Biol. 2000; 130: 155-64), electroporation (Heiser W C. Methods Mol Biol. 2000; 130: 117-34), or recombinant viruses (Walther W. Stein U; Drugs 2000 August; 60 (2): 249-71).

Techniques for expressing albumin in microorganisms, particularly yeast, and for purifying it from the culture medium are disclosed in U.S. Pat. No. 5,637,504, U.S. Pat. No. 6,034,221 and WO 00/44772, all of which are incorporated herein by reference.

Therefore, the invention further provides an expression cassette comprising a promoter operably linked to a nucleotide sequence as described herein encoding a mutant albumin as described herein. Nucleotide sequences encoding serum albumins, which may be mutated in accordance with the present invention, are also readily available from the Genbank database.

In addition, the invention provides a pharmaceutical composition comprising a mutant albumin as described herein and a pharmaceutically acceptable carrier.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

The mutant albumins of the present invention may be provided as pharmaceutical formulations wherein the mutant albumin is admixed with a pharmaceutically acceptable carrier (e.g. binder, corrective, corrigent, disintegrator, emulsion, excipient), diluent or solubilizer to give a pharmaceutical composition by a conventional manner, which is formulated into, for example, tablet, capsule, granule, powder, syrup, suspension, solution, injection, infusion, deposit agent, suppository and administered for example orally or parenterally.

When the tablets are used for oral administration, typically used carriers include sucrose, lactose, mannitol, maltitol, dextran, corn starch, typical lubricants such as magnesium stearate, preservatives such as paraben, sorbin, antioxidants such as ascorbic acid, α-tocopherol, cysteine, disintegrators or binders. When administered orally as capsules, effective diluents include lactose and dry corn starch. A liquid for oral use includes syrup, suspension, solution and emulsion, which may contain a typical inert diluent used in this field, such as water. In addition, sweeteners or flavours may be contained.

In the case of parenteral administration such as subcutaneous injection, intravenous injection, intramuscular injection, intraperitoneal injection or infusion, the pH of the active ingredient solution may be appropriately adequately adjusted, buffered or sterilized. Examples of usable vehicle or solvent include distilled water, Ringer water and isotonic brine. For intravenous use, the total concentration of solute is adjusted to make the solution isotonic.

Suppositories may be prepared by admixing the compounds of the present invention with a suitable nonirritative excipient such as those that are solid at normal temperature but become liquid at the temperature in the intestine and melt in rectum to release the active ingredient, such as cocoa butter and polyethylene glycols.

The dose can be determined depending on age, body weight, administration time, administration method, combination of drugs, the level of condition for which a patient is undergoing therapy, and other factors. While the daily dose may vary depending on the conditions and body weight of patients, the species of active ingredient, and administration route, in the case of oral use, the daily dose may be about 0.1-100 mg/person/day, preferably 0.5-30 mg/person/day. In the case of parenteral use, the daily dose may desirably be 0.1-50 mg/person/day, preferably 0.1-30 mg/person/day for subcutaneous injection, intravenous injection, intramuscular injection and intrarectal administration.

The mutant albumins of the present invention may be of use for example in human or animal medicine for the treatment of deficiency diseases and infections, treatment of metal overload and/or for conditions where control of metal concentrations may be linked to the physiological function of either another metal ion or an organic molecule, such as a drug or natural molecule.

It may also be possible to regulate the amount of a metal, such as zinc, present in blood using the mutant albumins of the present invention, or facilitate treatment of a subject displaying problems with zinc absorption. Moreover, mutant albumins which display particularly strong metal binding affinity may be used in biosensors to detect metals in an environment.

Additionally observations that the zinc bound to the albumin may be in the form of Zn²⁺ which may bind chloride ions, also leads to the possibility that albumin with bound zinc may be used as a chloride sensor and access to the Zn could be regulated by blood chloride concentration (this might also control catalytic activity).

The present inventors have also observed that mutant albumins according to the present invention have effects on cell growth in culture. The mutants can have an effect on the distribution of cells bound to a substrate and those found in the medium. It has also been observed that some mutants e.g. Asn99Asp can lead to overall increased cell growth. The present invention therefore also relates to the method or use of mutant serum albumins according to the present invention to alter growth characteristics of cells in culture. The alteration in growth characteristics can include changes in adhesion, percentage viability and/or cell growth e.g. titre, cell distribution between those substrate adhered and those found dispersed in medium, or differences between dead or viable cells adhered or in the medium.

Albumin is commonly included in cell culture media, especially media for mammalian cell culture and particularly serum-free media. The medium to which the modified albumin of the invention is added may or may not contain copper, zinc and/or cadmium. Suitable examples include Eagles' medium, Dulbecco's modified Eagle's medium (Dulbecco's minimal medium), Ham's F10 and F12 media, Iscove's modified Dulbecco's medium and RPMI media. In the case of such media that normally contain albumin, the modified albumin of the invention may be substituted partly or wholly for the native albumin (human or bovine) or may be added to an amount in excess of the normal amount of albumin. In the case of such media that do not normally contain albumin, the modified albumin of the invention may be added.

The cells for which the medium is used may be any animal cells, particularly avian (such as chicken) or mammalian cells, such as human, other primate (such as monkey), or rodent (such as hamster, rat or mouse) cells. The cell type may be derived from any tissue, for example the kidney, ovary or liver, and may be endothelial, epithelial, dermal, neural, lymphocytic, stem cell and the like. It may also be an artificial cell such as a hybridoma. Examples of suitable cells include tumorigenic or non-tumorigenic human hepatocytes, B lymphocytes, hybridomas, baby hamster kidney cells, Chinese hamster ovary cells and human embryonic kidney cells. The cells may be cultured on surfaces, such as vessel walls, porous matrices or beads, or they may be suspended freely in the medium.

The cultured cells may be used to produce any substance that is naturally produced by the particular cell, or they may be engineered to express other products, such as therapeutic proteins. Examples include monoclonal antibodies and analogues thereof (such as single chain variable region fragments and humanized IgG kappa light chains), blood clotting factors (such as Factors VII, VIII, XI and XIII), anti-thrombin III, cytokines (such as interleukins, for example interleukin-2, and interferons, such as interferon-αor interferon-γ), growth factors (such as insulin-like growth factor), thrombomodulin, glutamine synthetase, prourokinase and plasminogen.

The modified albumins of the invention may be included in tissue culture media prepared for prokaryotes and yeast, as well as cultured cells and tissues derived from vertebrates and invertebrates to produce a desired effect on the cells, such as increased adherence, growth and/or expression and secretion.

It is within the ordinary skill in the art to determine an appropriate concentration of an inventive modified albumin in a selected culture medium. In one embodiment, the modified albumin is introduced into a cell culture system at a concentration of about 50 μM to about 30 mM. In a further embodiment, the peptide is introduced into a cell culture system at a concentration of about 250 μM to about 20 mM. Moreover, multiple modified albumins may be added to a culture medium surface to produce a synergistic effect (if those have the same effect on the cells) or to produce multiple effects (if each modified albumin has a different effect on the same cells).

The modified albumins of the invention which increase cell adhesion may be dissolved in a carrier such as water to produce a solution for coating tissue culture substrate or other surfaces for growth of anchorage-type cells. For example, a solution containing one or more said modified albumins of the invention may be distributed onto a surface and dried in a reverse airflow hood that results in said modified albumins being present on the surface in the form of a dried film.

The mode of attachment of said modified albumin, of the invention to a surface includes non-covalent interaction, non-specific adsorption, and covalent linkages. In one embodiment of the invention, the albumins may be adsorbed directly to a surface. In a further embodiment, the peptide may be adsorbed to a surface which has already been precoated with, but is not limited to, at least one of the following: keyhole limpet haemocyanin, collagen, fibronectin, laminin, polylysine, a peptide having a cell-surface receptor recognition sequence, an immunoglobulin, a polysaccharide, or a growth factor. In another embodiment, the albumin and one of the proteins described above are applied simultaneously, either free or as a conjugate to the surface.

The growth enhancing modified albumins of the present invention which are suitable for promoting adherence and/or growth of a variety of anchorage-dependent cells on surfaces, including two dimensional or three dimensional surfaces. For example, the surface may be that of a bioreactor which allows cells to attach in 3-D arrays. More efficient bioreactors than presently exist can be designed by attaching the cells to 3-D surfaces modified with the inventive peptides.

With specific reference to the types of surfaces which may be used in the practice of the present invention, suitable surfaces would include, but are not limited to, ceramic, metal or polymer surfaces. Most desirably, the present invention is used in the treatment of polymer surfaces and ceramic, e.g. glass surfaces. Suitable surfaces for use in the present invention, include, but are not limited to, plastic dishes, plastic flasks, plastic microtitre plates, plastic tubes, surtures, membranes, films, bioreactors, and microparticles. Polymer surfaces may include, but are not limited to, poly(hydroxyethylmethacrylate), poly(ethylene terephthalate), poly(tetrafluoroethylene), fluorinated ethylene, poly(dimethyl siloxane) and other silicone rubbers. Glass surfaces may include glycerol propylsilane bonded glass.

There is also provided a cell culture medium comprising a mutant serum albumin according to the present invention.

### DETAILED DESCRIPTION OF THE INVENTION

The present invention will now be further described by way of example and with reference to the figures, which show:

FIG. 1 shows a model of the three dimensional structure of human serum albumin as reported in PDB 1AO6, with the metal binding site identified herein, highlighted;

FIG. 2 shows in more detail amino acid side-chains located in and around the proposed zinc binding site;

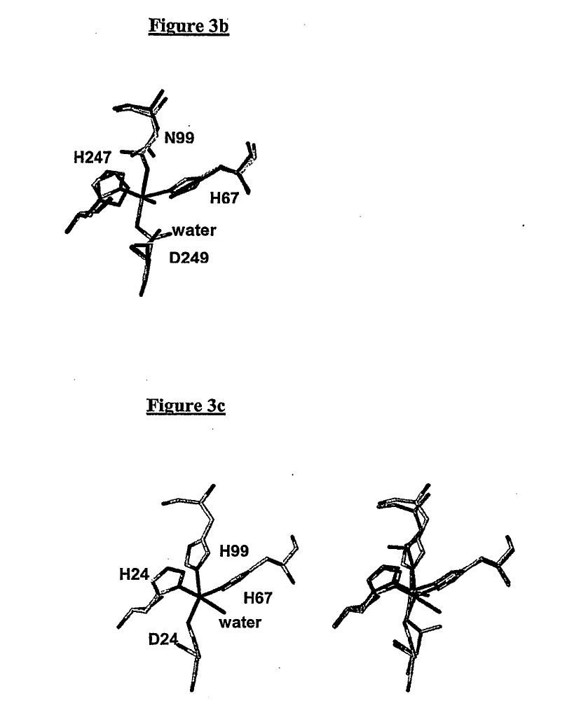

FIG. 3a shows an initial model of zinc site in wild-type albumin, in comparison with apo-rHA (1AO6).

FIG. 3b shows recalculated, improved model of a zinc site in wild-type human serum albumin, in comparison with apo-rHA (1AO6). Force-field energy of the zinc site: 59.1 kcal/mol;

FIG. 3c shows model for the metal site in the Asn99His mutant, in comparison with wild-type Zn rHA (green). Force field energy for the zinc site is 83.2 kcal/mol.rmsd to wild-type apo: 0.54 Å; to wild-type Zn-albumin: 0.56 Å;

FIG. 3d shows model for the metal site in the Asn99Asp mutant;

FIG. 3e shows inter-domain H bonds at the potential zinc site in models of zinc-free wild-type and mutant albumins. a: Wild-type; b: Fatty-acid loaded wild-type; c: Asn99His mutant model; d: Asn99Asp mutant model;

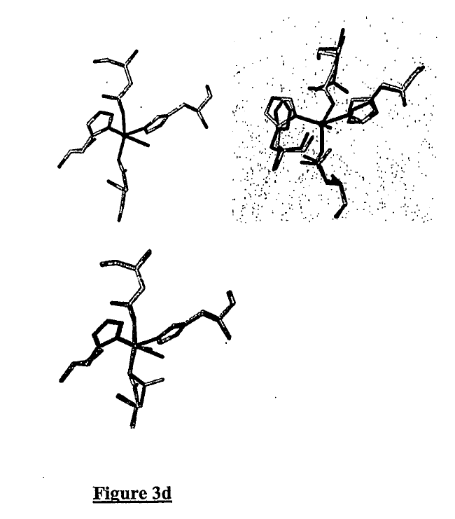

FIG. 4 shows circular dichroism spectra of wild type (solid line), and H67A (dashed line) albumin;

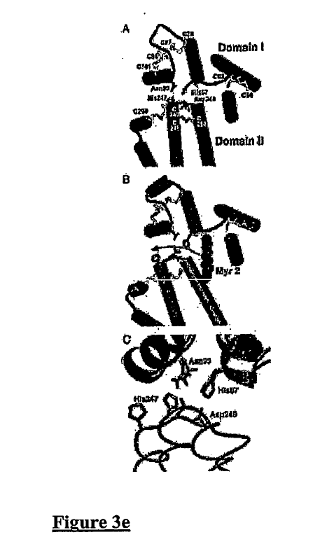

FIG. 5 shows ¹¹¹Cd NMR of native and H67A rHA with 2 mol equivalent of ¹¹¹CdCl₂;

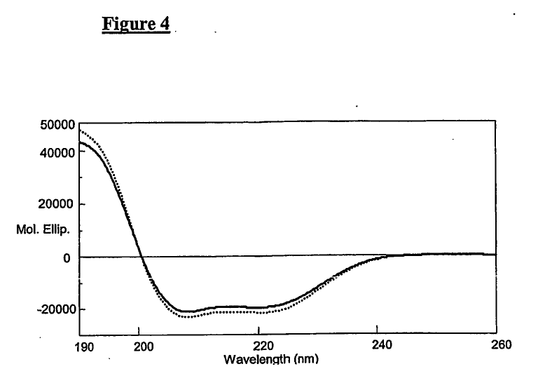

FIG. 6 shows ¹¹¹Cd NMR of rHA with 2 mol equivalent of ¹¹¹CdCl₂ in the presence of a) zinc and b) copper;

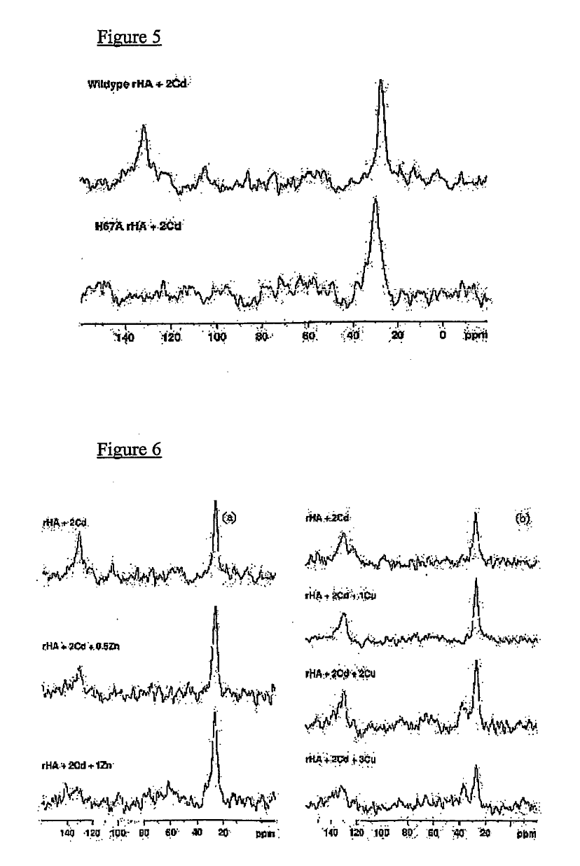

FIG. 7 shows UV-visible absorption spectra of (a) native rHA and (b) H67A rHA with 0.2 to 2 mol equivalent of CuCl₂ in 0.2 mol equivalent steps (bottom to top);

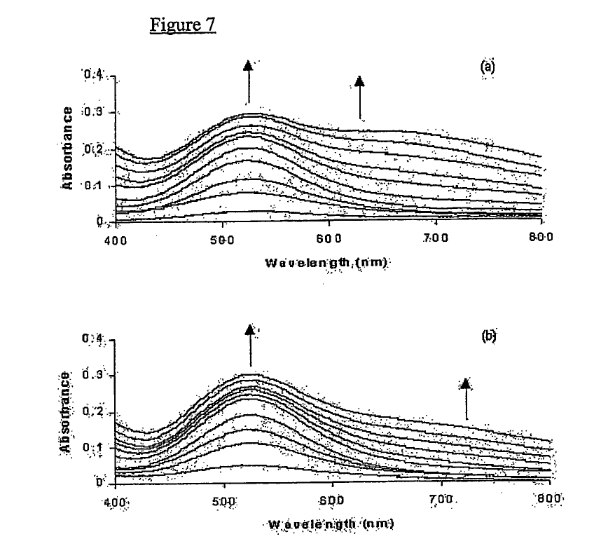

FIG. 8 shows the potential zinc binding site in an asn99asp mutant without zinc bound. shown in magenta on the right side overlay is the wild-type structure. the force-field energy of the mutated site (101.4 kcal/mol) is insignificantly higher than that of the wild-type (55.6 kcal/mol) and the asp99his site (75.6 kcal/mol).

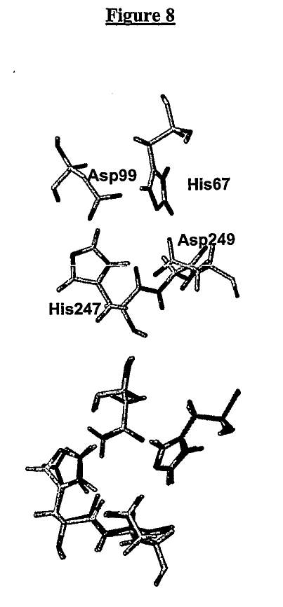

FIG. 9 shows the 1D ¹¹¹Cd NMR spectra of recombinant albumins (wild-type and Asn99His mutant) with 2 mol equivalents of ¹¹¹Cd²⁺ (conditions: 1 mM protein, 50 mM Tris-Cl, 50 mM NaCl, 295 K);

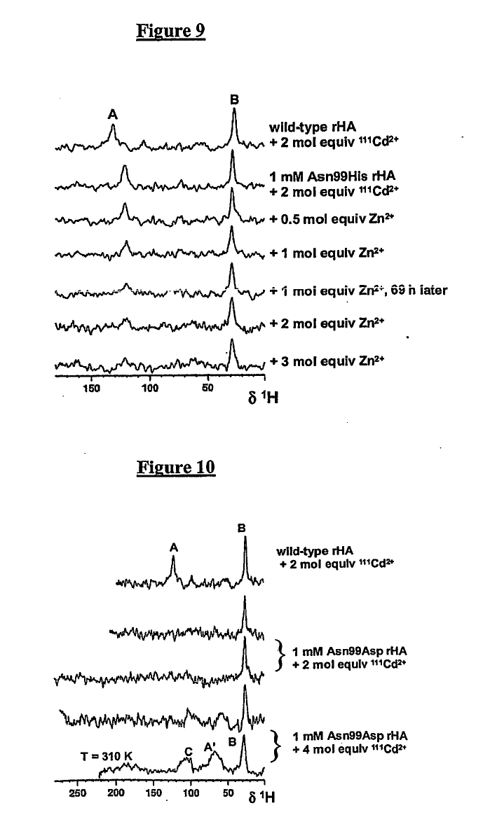

FIG. 10 shows the 1D ¹¹¹Cd NMR spectra of recombinant albumins (wild-type and Asn99Asp mutant) with 2 mol equivalents of ¹¹¹Cd²⁺ (conditions: 1 mM protein, 50 mM Tris-Cl, 50 mM NaCl, 295 K if not stated otherwise);

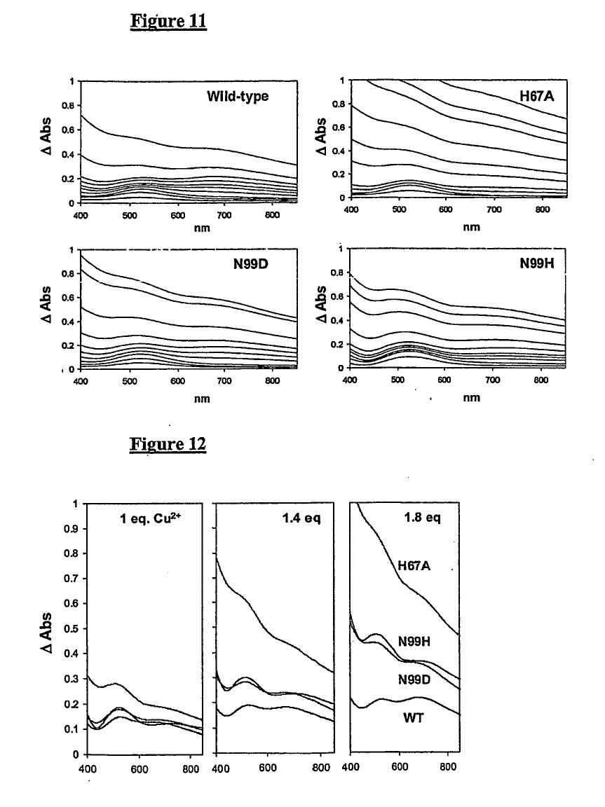

FIG. 11 shows the titrations of 1 mM rHA with copper(II) (pH 7.4, 0.2 M potassium phosphate). CuCl₂ was added in 0.2 mot equiv portions in each case. Shown are difference spectra, corrected for the absorption of albumin;

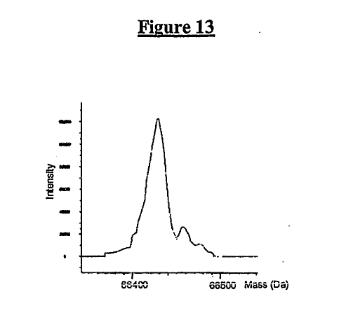

FIG. 12 shows the direct comparison of the effect various amounts of Cu²⁺ on the UV-Vis difference spectra of wild-type and mutant albumin.

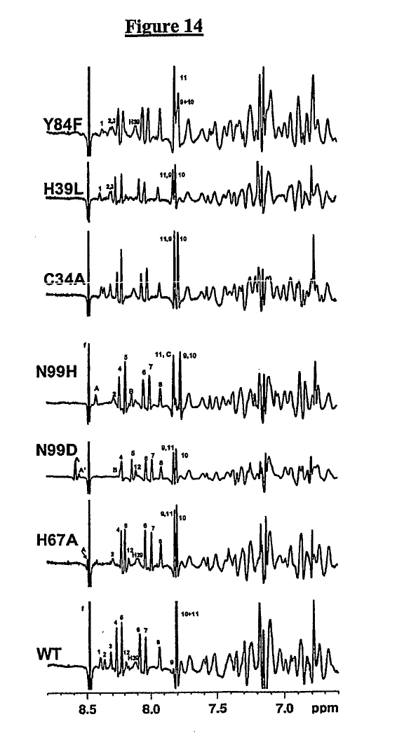

FIG. 13 shows deconvoluted FT-ICR-MS spectrum of wild-type rHA (20 μM in 8 mM NH₄Ac, 25% methanol, 1% acetic acid). Note the narrow line shape (half-height width ca. 25 Da) which enables the detection of small-molecule adducts;

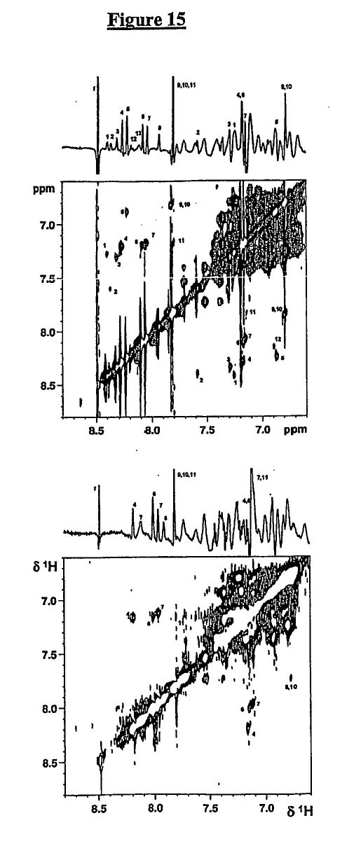

FIG. 14a shows a survey of resolution-enhanced 1D ¹H NMR spectra of recombinant albumin mutants. FIGS. 14b,c,d, and e show portions of 2D TOCSY NMR spectra of wild-type, His67Ala, Asn99His, and Asn99Asp rHA, respectively, showing His H82/Hε1 cross-peaks. All samples were 1 mM in 50 mM Tris-Cl, 50 mM NaCl, and all experiments were carried out at 310 K. pH values vary between 7.28 (N99H) and 7.40 (H67A), which accounts for slight differences in chemical shifts for individual protons. Observable Hε1 protons are labelled with numbers, f denotes formate, which had been added as a chemical shift standard;

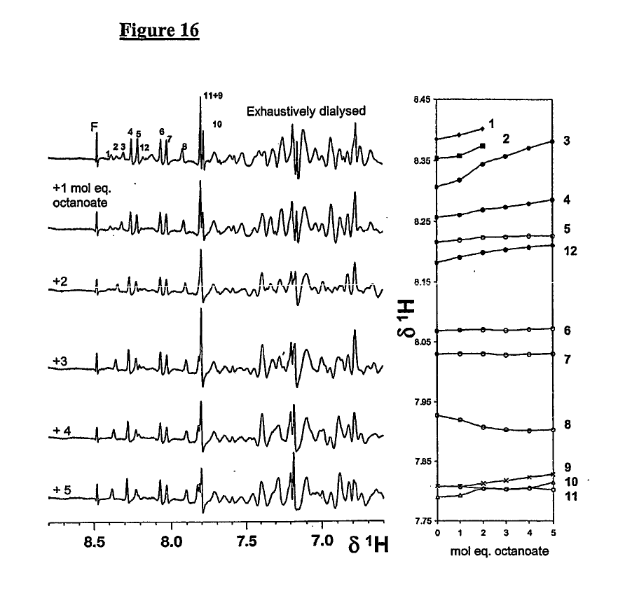

FIG. 15 shows portions of 1D and 2D TOCSY spectra with 1 mol equiv Zn²⁺ added (pH*=7.37) showing histidine Hδ2/Hε1 cross-peaks;

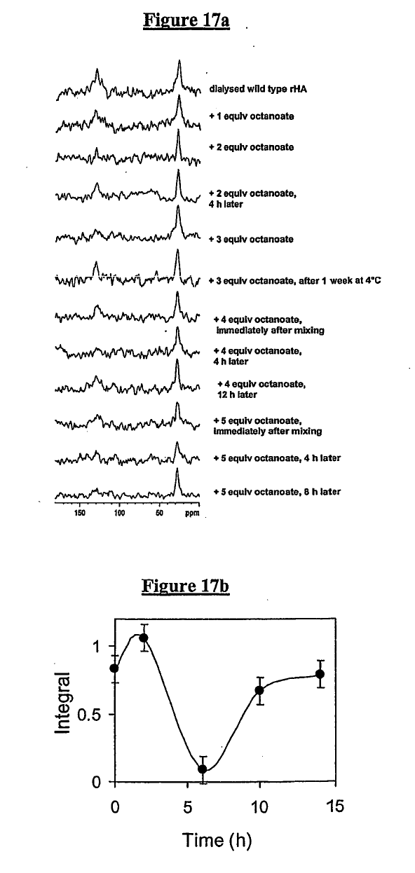

FIG. 16a shows portions of resolution enhanced 1D NMR spectra of wild-type rHA (1 mM in 50 mM Tris-Cl, 50 mM NaCl, pH*=7.26) with varying amounts of octanoate; FIG. 16b shows the effect of increasing amounts of octanoate on chemical shifts of histidine Hε1 protons;

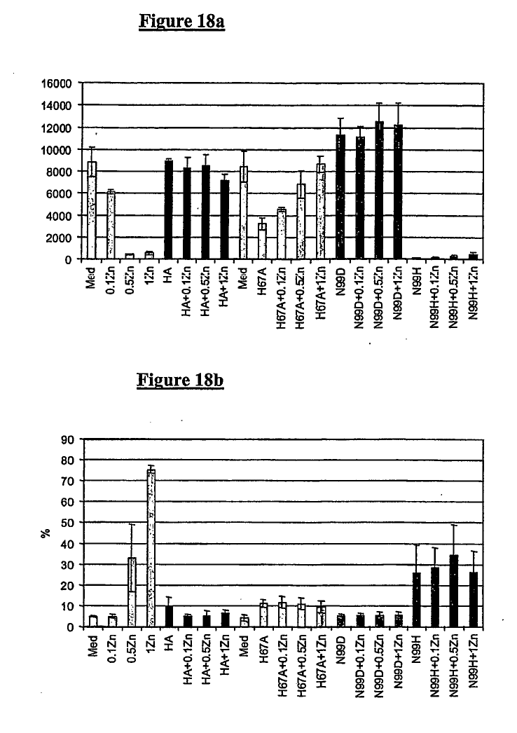

FIGS. 17a & b show titration of Cd₂rHA with octanoate. Conditions: 1 mM rHA, 2 mol equiv ¹¹¹CdCl₂, 50 mM Tris-Cl, 50 mM NaCl, 10% D₂O, pH 7.1, 298 K, 10 mm BBO probe. The acquisition of one spectrum typically takes 4 hours. The graph in 17b shows the time-course for 4 equivalents [As the acquisition of each spectrum takes 4 h, the mid-point (i.e. two hours after starting the experiment) of each spectrum has been taken as the average time-point];

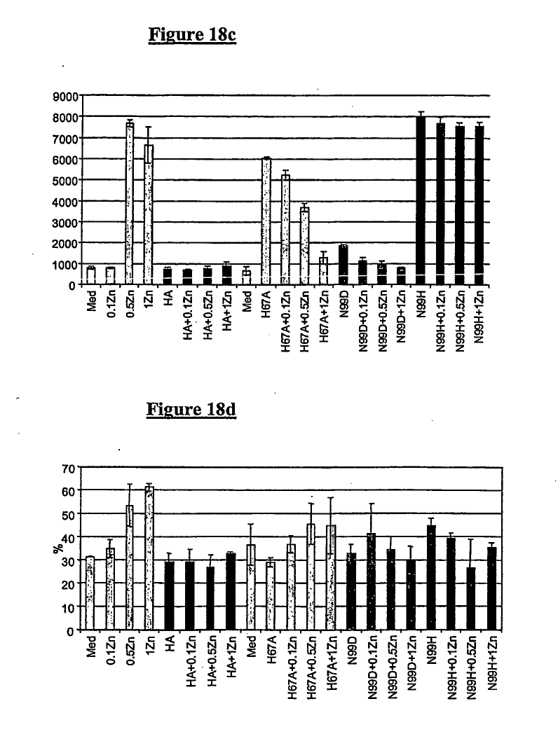

FIG. 18a shows cell counts in layer using native and mutant serum albumins;

FIG. 18b shows percentage of dead cells in layer using native and mutant serum albumins;

FIG. 18c shows cell counts in medium using native and mutant serum albumins; and

FIG. 18d shows percentage of dead cells in medium using native and mutant serum albumins.

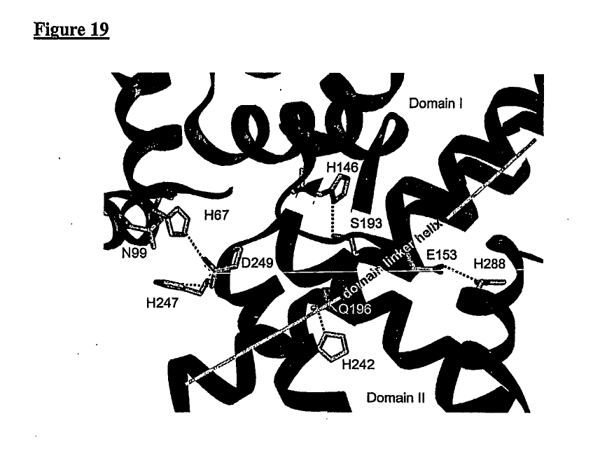

FIG. 19 shows mutation identified as being involved domain I-domain II contacts via H bonds.

### MATERIALS AND METHODS

a) Molecular Modelling

A published crystal structure at 2.5 Å of unliganded (apo)albumin (PDB accession code 1AO6) was used throughout as starting point for the models. The models were built in Sybyl (TRIPOS Inc., Version 6.8) and submitted to energy minimisation in order to optimise geometry. It was noted that in previous runs the program had ignored the presence of disulfide bonds, which had led to hydrogen atom addition and breakage of the disulfide bonds. This problem, which slightly influences overall protein structure, but not the zinc site itself, has now been corrected in the final modelling runs. In the minimisations, the TRIPOS force field was used, after specific force-field parameters for zinc had been implemented. Bond lengths for Zn²⁺ bound to histidine (2.00 Å), aspartate or glutamate (2.00 Å), and water (2.06 Å) were taken from Harding, M. M. Acta Cyst. D57, 401-411 (2001), http://tanna.bch.ed.ac.uk. These values also agree with results from analyses of the pdb via the metalloprotein database (Castagnetto, J. M., Hennessy, S. W., Roberts, V. A., Getzoff, E. D., Tainer, J. A., Pique, M. E., Nucleic Acids Res., 30, 379-382 (2002). Force constants were taken from the TRIPOS force field. Bond angles around zinc were not constrained. In a first step, the geometry around Zn²⁺ was optimised by 100 steps of energy minimisation taking into account only the Zn²⁺ ion, the four protein ligand residues, and the water molecule. A further 50 steps of energy minimisation were then applied to residues 65-69, 97-101, and 247-251, and the Zn²⁺ ion and the water molecule, to remove bad geometries and van der Waals contacts which had been introduced through atom movements in the first step. Finally, 30 more steps were applied to the entire protein for the same reason. The overlays in the figures were generated in Sybyl with the “Fit monomers” routine, which also supplies the rmsd values. For the modelling of the mutant zinc-free albumins, the N99 side-chain was mutated in silico to the desired side-chain (Asp or His), and possible bad contacts were relieved by applying 30 steps of energy minimisation to the whole molecule. For the zinc-containing mutant models, the same approach employed for the wild-type model was used, exploring several possible starting structures with different metal-to-ligand connectivities.

Computer Programs and Databases

Sequence alignments were carried out using ClustalW, European Bioinformatics Institute (www.ebi.ac.uk/clustalw/) with sequences obtained from Entrez Protein, National Centre for Biotechnology Information (www.ncbi.nlm.nih.gov/entrez/). 3-dimensional coordinates for human albumin (PDB 1AO6) were obtained from the Brookhaven Protein Databank (www.rcsb.org/pdb/).

Site-Directed Mutagenesis

Oligonucleotide-directed mutagenesis was used to prepare cDNAs encoding the H67A mutated form of albumin. The mutagenic oligonucleotides 5′-GCTGAAATTGTGACAAATCACTTGCTACCCTTTTTGGAGACAAATTATGC-3′ and 5 ′GCATAATTTGTCTCCAAAAAGGGTAGCAAGTGATTTGTCACAATTTTCA GC-3′ were supplied by Delta Biotechnology Ltd., Nottingham. Mutagenesis was performed using the QuikChange® Site-Directed Mutagenesis Kit (Stratagene). A clone containing the desired mutation was identified by nucleotide sequence analysis across the mutation site by dideoxy chain termination sequencing. The mutated cDNA was inserted into a PUC9 yeast expression vector and transformed into Saccharomyces cerevisiae cells by electroporation.

Expression and Purification

The S. cerevisiae cell cultures, following growth at 30° C. for 4 days were centrifuged at 3,000 rpm for 30 min. The supernatants were then removed and filtered. The recombinant protein was concentrated from the supernatant, using cation-exchange chromatography. An SP-sepharose Fast Flow cation-exchange column (column volume=225 mL) was equilibrated with 4 column volumes of a 30 mM sodium acetate buffer, pH 5.5. The filtered supernatants were split into two batches of approximately 3 L. Sodium octanoate was added (7.5 mL of a 2 M solution) to each batch and the pH was adjusted to 4.5 with acetic acid and the conductivity was adjusted to 5.5 mS cm⁻¹ with deionised water before loading onto the column. After loading, the column was then washed with 8 column volumes of 50 mM acetate, 8 mM NaOH, pH 4.0 and 4 column volumes of a 27 mM sodium acetate buffer containing 2 M NaCl, pH 4.0. A third wash was carried out with 10 column volumes of the equilibration buffer. Finally the column was eluted with 2 column volumes of 85 mM sodium acetate containing 5 mM octanoic acid, pH 5.5.

SP-sepharose Fast Flow eluents were then further purified by anion-exchange chromatography on a DEAE fast flow column (column volume=167 mL). The column was equilibrated with 15 column volumes of 30 mM acetate, 27 mM NaOH, pH 5.5. The conductivity of SP-sepharose eluents was adjusted to 3.0 mS cm⁻¹ with deionised water before loading onto the column. After loading the column was then washed with 5 column volumes of 15.7 mM K₂B₄O₇.4H₂O, pH 9.2. The column was eluted with 0.75 column volumes of 85 mM acetate, 110 mM K₂B₄O₇.4H₂O, pH 9.4.

DEAE eluents were then purified further by affinity chromatography on Delta Blue Agarose (Prometic Biosciences) column (column volume=423 mL). The column was equilibrated with 2 column volumes of a 250 mM ammonium acetate buffer, pH 8.9 before loading the DEAE eluent. After loading, the column was then washed with 5 column volumes of the equilibration buffer. The column was eluted with 2 column volumes of 50 mM phosphate buffer containing 2 M NaCl, pH 6.9.

Delta Blue eluents were then concentrated using a 10 kD MWCO Pall Filtron LU Centramate filter connected to a peristaltic pump. It was determined that 4.25 g of H67A albumin was recovered. A sample of concentrated solution from the purified product was diluted to 5 mg mL⁻¹ and 10 μL was applied to an SDS-PAGE gel. The gels were made and ran using the standard method of Laemmli (1970) Nature 227, 680-685. The gel was stained with both coomassie blue stain and silver stain and revealed no other proteins to be present at the 1% level (therefore protein is approximately 99% pure).

Circular Dichroism

Native recombinant human albumin (rHA) (Delta Biotechnology Ltd., Nottingham) and the H67A mutant albumins were diluted to approx. 1.5 mg mL⁻¹ in 200 mM potassium phosphate, pH 7.4. Spectra were recorded for both of the proteins. The instrument used was a JASCO J-600 spectropolarimeter. Secondary structure estimations were calculated using the SELCON procedure.

1D ¹¹¹Cd—{¹H} NMR spectra (106.04 MHz, Bruker DMX500) were routinely acquired using a 10 mm BBO (direct observe) probe head at 295 K and 0.1 M Cd(ClO₄)₂ (0 ppm) as external standard. Proton decoupling was achieved by composite pulse decoupling using GARP. Protein samples were generally in 50 mM Tris, pH 7.1, 100 mM NaCl, 10% deuterium oxide with 2 mol equiv of ¹¹¹CdCl₂. ¹¹¹CdCl₂ was generated by dissolving ¹¹¹CdO (95.11% isotopic purity, Oak Ridge National Laboratory, Tennessee, USA) in the appropriate amount of 1 M HCl.

¹¹¹Cd-NMR studies were carried out using 1.5 mM rHA or His67Ala mutant protein at the same concentration. Various equivalents of ZnCl₂ or CuCl₂ were added for metal titration experiments, the pH was checked and adjusted (if required) after each addition. Spectra were acquired over a sweep width of 30 kHz (280 ppm) into 4 k complex data points, with a ¹¹¹Cd pulse width of 17.5 μs (90°), 36 k transients, an acquisition time of 0.10 s, and a recycle delay of 0.30 s. Prior to Fourier Transformation, data were zero-filled to 16 k data points and apodized by exponential multiplication (120 Hz line broadening).

¹¹¹¹Cd-NMR studies on Asn99Asp and the Asn99His mutant rHA were carried out using 1 mM solutions of mutant albumins. Most spectra were acquired over a sweep width of 32 kHz (300 ppm) into 8 k complex data points, with a ¹¹¹Cd pulse width of 17.5 μs (90°), 36 k transients, acquisition time of 0.13 s, and a recycle delay of 0.24 s. Prior to Fourier transformation, data were zero-filled to 32 k data points and apodized by exponential multiplication (150 Hz line-broadening).

d) ¹H NMR Spectroscopy

To eliminate NH resonances, lyophilised samples were dissolved in D₂O (99.9% isotopic purity, Aldrich) at ca. 50 mg/mL, kept at 295 K for 48 h, and were lyophilised again, and then dissolved to yield 1 mM solutions in D₂O containing 50 mM NaCl, 50 mM Tris. Sodium formate was added at a concentration of 1 mM as internal calibration standard (8.48 ppm relative to sodium 3-(trimethylsilyl)propionate; TSP). The pH* (pH meter reading) was adjusted to 7.3-7.4, corresponding to a pH of 6.9-7.0 (Glasoe, P. K. and Long, F. A. J. Phys. Chem., 64, 188 (1960) 1D and 2D ¹H NMR experiments were routinely carried out at 310 K on a Bruker Avance 600 spectrometer operating at 599.82 MHz using a Z-gradient triple-resonance (¹H, ¹³C, ¹⁵N) probe head. Typically, 512 transients were acquired for the 1D spectra (90° excitation pulse, 9 kHz sweepwidth, 8 k time domain data points) using a simple presaturation pulse sequence for residual water suppression (1.5 s relaxation delay).

The data were zero-filled to 32 k, apodized with an optimised combination of squared sine bell and Gaussian functions for resolution enhancement, and Fourier transformed. For 2D TOCSY experiments (90° excitation pulse, 8.4 kHz sweepwidth, mixing time 65 ms, 1.3 s relaxation delay), 48 or 56 transients for each of 2×512 t¹ increments (hypercomplex acquisition using time-proportional phase incrementation (TPPI)) were acquired into 4k complex data points, using a sensitivity-enhanced, double-pulsed field-gradient spin-echo sequence for residual water suppression. The data were apodised using squared sinebell functions, and the real Fourier transform was carried out on 2 k×2 k data points.

Some spectra for the wild-type were also recorded in 0.1 M potassium phosphate (KHP) buffer. It was noted that the chemical shifts of the histidine Hε1 protons are also dependent on the identity of the buffer. Generally, signals are shifted upfield in KHP buffer compared to spectra taken at the same pH* (pD taken in D₂O) in Tris buffer. The quality of the spectra is similar, and a protein concentration of 1 mM has been found to be optimal for most experiments carried out. Above this concentration, the solutions become too viscous, which appears to be disadvantageous for shimming as well as for the line widths of the signals, and makes handling of the solutions (e.g. pH adjustment, mixing with reactants) difficult.

e) UV-Vis Spectroscopy: Copper Titrations

Albumin samples were 1 mM or 2 mM in 200 mM potassium phosphate, pH 7.4. From a 700 mM CuCl₂ stock solution, 0.2 μl aliquots (corresponding to 0.2 mol equivs.) were successively added. The sample was thoroughly mixed, and UV-Vis spectra were recorded using a Shimadzu UV250 IPC spectrophotometer between 400 to 800 nm after 5 min. Initially, solutions turned pink, whereas the later additions led to clouding, which accounts for the overall increase in absorption observed in the spectra. The onset of clouding (formation of Cu₃(PO₄)₂) clearly differs for the various albumin mutants.

f) Cell Culture Experiments to Assess Cytotoxicity

During the inventors' studies, two approaches to explore zinc and albumin cytotoxicity were developed. The approach used in initial experiments relied on the determination of cell viability with Trypan Blue, the second approach employs FACS (Fluorescence-activated cell sorting, a flow-cytometric application), after dead cells have been stained by propidium iodide. The second approach has several advantages; it is more efficient with respect to both time and material consumption.

i) Standard Culture Conditions

WRL-68 cells were cultured in DMEM (Dulbecco's modified eagles medium) supplemented with 10% FCS (newborn calf serum), penicillin and streptomycin and ×1 concentrate NEAA (Non-essential amino acids). Cells were grown in 80 cm² tissue culture grade flasks at 37° C., 5% CO₂ in an incubator. Cells were supplemented with fresh medium every 2-3 days or as required by monitoring the bicarbonate colour indicator in the medium, with a yellow colour indicating supplementation was necessary. Once cells grown in flasks became confluent they were harvested using Trypsin+EDTA and PBS. The cell suspension was centrifuged at 1000 rpm for 10 minutes in a MSE Mistral 1000 centrifuge until a pellet was formed. The supernatant was removed and the pellet of cells resuspended in medium and used as required.

ii) Cell Viability Counts Using Trypan Blue

Trypan blue is used to estimate the proportion of viable cells in a population. The reactivity of the stain is based on the fact that the chromophore is negatively charged and does not react with the cell unless the membrane is damaged. Live (viable) cells do not take up the dye and dead (non-viable) cells do.

Cells were typically seeded using 0.5 ml at 15.2×10⁵ cells/ml (need to check this value with Kerry Bunyan) into small cell culture flasks. These were then left overnight to equilibrate. Medium was removed and the cells washed with PBS.

Cells were then treated with recombinant human albumin-(rHA) alone (40 mg/ml), H67A human albumin (H67A) (40 mg/ml), rHA and h67A with 0.1, 0.5 and 1.0 molar equivalents Zn and with Zn alone at the same concentrations. All treatments were made up in supplemented DMEM. Controls were also set up were medium alone was added. Flasks were left for two nights following treatment. Following incubation with albumin and zinc the medium was removed and kept for analysis. The cell layer was then washed twice with PBS and this wash was added to the medium collected. The cell layer was then removed from flasks using Trypsin+EDTA. Again the flasks were washed with PBS and this was added to the cell suspension. All samples of medium and cell suspension were then centrifuged.

Once centrifuged the supernatant was aspirated off and the pellet resuspended in PBS. To estimate the concentration of viable cells and total cell numbers in the collected medium and cell layer, 200 μl the well mixed sample, 300 μl PBS and 500 μl of 0.4% trypan blue (Sigma) solution were mixed and left at room temperature for a 2-3 minutes. The suspension was transferred to a haemocytometer, viewed using an Olympus inverted phase-contrast light microscope and the number of dead (blue) and live (colourless) cells were counted within the 4×4 square grid. Counting was made of 10 square grids in total. These cell counts were used to estimate the total number of cells and number of live cells (viability) according to the following equations:
cells/ml=average cell count×5 (dilution factor)×1×10⁴ (haemocytometer chamber factor)
Viable cells/ml=number of live cells×5 (dilution factor)×1×10⁴ (haemocytometer chamber factor)
cell viability (%)=(total viable/total viable and nonviable)×100
These figures were then displayed in graphical form to show total number of cells found in the medium and cell layer and viability of these cells.
iii) Analysis via Flow Cytometry and Fluorescence-Activated Cell Sorting (FACS)

Cells were plated onto 12 well plates at 0.0995×10⁶ cells/ml using 0.5 ml per well. Plates were then left overnight to equilibrate. The medium was then aspirated off and the cell layer washed with PBS. Subsequently the cells were treated with medium supplemented with 0, 60, 300 or 600 μM ZnCl₂, in the absence or presence of wild-type albumin, or His67Ala, Asn99Asp, or Asn99-H is mutant albumin. Cells with medium alone were used as controls. Following 48 hours incubation the plates were then analysed using flow cytometry. For this the medium was removed and the cell layer washed twice with PBS. This wash was added to the medium that had been removed. The cell layer was then removed using Trypsin+EDTA and washed twice with PBS and these washings added to the cell suspension. All samples had 10% FCS added prior to cell sorting. Propidium iodide (1 μg/ml) was added to samples immediately prior to counting to detect cell death. Samples were then run using a Beckman Coulter EPICS cell counter. Total number of events after 60 seconds was recorded to determine cell numbers for comparison between groups.

### EXAMPLES

Identification of Zinc Binding Site by Molecular Modelling

NMR studies have revealed that ¹¹³Cd chemical shifts upon binding to albumin suggest metal coordination to the protein at 2 sites. (Sadler and Viles (1996) Inorg. Chem. 35, 4490-4496). At the site where Zn²⁺ displaces Cd²⁺ the chemical shift is in the range for coordination of the metal to the protein to involve 2-3 imidazole nitrogens (Oz et al. (1998) Biochem. Cell Biol. 76, 223-234).

The crystal structure coordinates of human albumin were obtained from the Brookhaven Protein Databank (PDB 1AO6) and were examined using WebLab viewer Pro v4.0 (Accelrys). Histidine residues were highlighted (since these are the main nitrogen donating residues in proteins for metal coordination) and distances between each were measured. The present inventors found that only one site on the molecule had present 2 histidine side-chains within 5 Å from each other. This led us to believe that His67 and His247 were involved in the zinc binding site. The identification of other residues around this site revealed that Asn99 and Asp249 were also within close enough proximity to provide oxygen ligands for metal binding. Asn99 could also potentially provide a nitrogen ligand from the amide group of its side chain.

A database (Harding (2001) Acta Cyst. D57, 401-411; http://tanna.bch.ed.ac.uk) of amino acid side-chains coordinating to metals in proteins revealed that 3 other proteins contain zinc bound to 2 His, 1 Asp and 1 Asn residues (human calcineurin, E. coli 5′-endonucleotidase and kidney bean purple phosphatase) further suggesting this to be a suitable site for zinc binding. See FIGS. 1 and 2, which show the predicted region of metal binding as determined by the present inventors.

Modelling of Zinc into Proposed Binding Site

An initial model of Zn-containing albumin was built based on the published crystal structure (pdb accession code 1AO6) using Weblabviewer (Accelrys). The zinc site was modelled as 5 coordinate containing Cl⁻ as the fifth ligand, since in our 1D ¹¹¹Cd NMR studies we have noticed that the shift of the resonance is dependent on the Cl⁻ concentration, which makes binding of chloride under physiological conditions highly likely. Water as a fifth ligand is another possibility.

The model was imported into Sybyl v6.8 (TRIPOS Inc.) for energy minimization to optimise geometry, using the TRIPOS force field, after some specific parameters for zinc had been defined. Bond lengths for Zn²⁺ bound to histidine (2.00 Å) and aspartate (2.00 Å) (and water, 2.06 Å) were taken from Harding (2001) Acta Cyst. D57, 401-411, and a bond length for an Asn-Zn²⁺ interaction (2.15 Å) was estimated based on the crystal structures of calcineurin, 5′-endonucleotidase and kidney bean purple acid phosphatase, which were obtained from the Brookhaven Protein Databank (pdb accession codes 4 KPB, 1AUI and 1TCO). The value for the Zn—Cl bond length was extracted from the Cambridge structural database (Allen and Kennard (1993) Chem. Design Autom. News 8, 31-37). Force constants were taken from the TRIPOS force field. Bond angles around zinc were not constrained at all, because for Zn²⁺ with a coordination number of 5, no regular or uniform angles are to be expected.

In a first step, the geometry around the zinc was optimised by 200 steps of energy minimisation of the zinc atom, the four protein ligand residues, and the chloride ion only. A further 10 steps of energy minimisation were then employed on the entire protein to remove bad geometries and Van der Waals contacts which had been introduced through the atom movements in the first step. The root mean square deviation (rmsd) values (which are an indication of structural difference) between the original protein structure and the modified model is 0.13 Å for all atoms, and 1.21 Å for the ligands residue atoms only.

FIG. 3 shows an overlay between the original structure (black) without hydrogens) and the present inventors model (grey) demonstrating that only relatively small movements were necessary to accommodate the zinc binding site. The site displays a distorted trigonal bipyramidal geometry with the two histidines in the axial positions. The chloride ligand points towards the outside of the protein. Additional modelling attempts with different starting structures furnished sites with similar geometries, but with the chloride ion on the opposite side of the Zn.

Attempts to model a tetrahedral site containing only the protein ligands yielded, despite applying angle constraints, a geometry resembling the distorted trigonal bipyramid found in the 5-coordinate model, with an empty equatorial binding site where the Cl⁻ had been.

Experimental Evidence to Support Modeling Theories

The present inventors expressed the mutant H67A in Saccharomyces cerevisiae cells and purified it to >95% by ion exchange and affinity chromatography. Circular dichroism revealed no major alterations in secondary structure between the H67A mutant and the wild-type protein (FIG. 4). ¹¹¹Cd-NMR studies on 1.5 mM recombinant human albumin (rHA), in 50 mM Tris, pH 7.1 with 2 mol equiv of ¹¹¹CdCl₂ confirmed binding at 2 sites (A and B) with peaks at 27 and 131 ppm (relative to Cd(ClO₄)), respectively. Under the same conditions the H67A mutant gave rise to a single peak at 29 ppm (FIG. 5). Addition of 0.5 and 1 mol equiv of ZnCl₂ to rHA in the presence of 2 mol equiv of ¹¹¹Cd²⁺ resulted in a decrease in intensity of the peak at 131 ppm (FIG. 6a). Addition of 2 and 3 mol equiv of CuCl₂ to rHA in the presence of 2 mol equiv of ¹¹¹CdCl₂ also appeared to affect Cd²⁺ binding at site B and led to the formation of a new ¹¹¹Cd peak at 37 ppm (FIG. 6b). The addition of 1 mol equiv of CuCl₂ did not affect Cd²⁺ binding. This is most likely due to the high affinity of Cu²⁺ for the N-terminus, with Cd²⁺ displacement occurring only after saturation of binding at this site. These results show that site B has a greater affinity toward Zn²⁺ than to Cd²⁺, that Cu²⁺ also binds competitively at this site, and also suggest the involvement of His67 for metal coordination.

Note also that FIG. 4 shows similar signs and magnitudes of circular dichroism bands for native, and H67A. This is indicative of H67A rHA having similar secondary structure to native albumin.

The number of nitrogen ligands coordinating to Cu²⁺ in peptides and proteins is known to affect the wavelength of the d-d absorption bands of these complexes. Aliquots of CuCl₂ were added to 2 mM solutions of rHA and the H67A mutant in 200 mM potassium phosphate, pH 7.4. An absorption band at 525 nm appeared after the first addition of CuCl₂, indicative of N-terminal loading of the proteins by Cu²⁺, characteristic of 4 N coordination to Cu²⁺. However a marked difference in absorption was observed after the further addition of 1 mol equiv CuCl₂ to each of the proteins. The native protein developed a second absorption band at 625 nm and the mutant a much broader band at 750 nm (FIG. 7). These bands suggest coordination of Cu²⁺ to 2 N and 1 N respectively (Pettit et al. (1990) J. Chem. Soc. Dalton. Trans. 3565-3570). This suggests that the His67 residue is important for Cu²⁺ binding as well as Zn²⁺, although does not provide information as to whether the Cu²⁺ ions still bind at this site (without the involvement of His67) or elsewhere on the protein.

Further Molecular Modelling of Metal Sites in Mutant Albumins

The present inventors have been able to improve modelling methodology, by optimising the energy minimisation protocol and by exploring different starting structures; therefore the inventors re-modelled the proposed Zn(II) site on wild-type albumin, to allow meaningful comparisons between the various models. In the following the results are summarised.

a) Wild-Type Albumin

The overall geometry of the new model does not significantly differ from the previous model (FIG. 3a), apart from the fact that we have now used water as a fifth ligand (all atoms rmsd: 0.05 Å; Zn site rmsd: 0.25 Å). The rmsd between the original structure (pdb entry 1AO6;[FIG. 3]) and the model is 0.67 Å for the zinc site atoms only, essentially suggesting that the zinc site in albumin is preorganised.

b) Modelling Studies were also Carried out on an Asn99His Mutant and Asn99Asp Mutant

Mutant models are shown in FIGS. 3c, 3d and 3c. FIG. 19 showing the proposed site in its metal-free form, demonstrates the effects of the mutations on inter-domain hydrogen bonds, which might play a role in conformational dynamics and allosteric interactions.

In summary, the modelling studies support the idea that mutant serum albumins can be produced which are capable of binding metals e.g. zinc at a different affinity with respect to wild type albumin and/or displaying other physiological characteristic(s).

Probing the Mutated Metal Site

The assessment of zinc binding in proteins can be difficult, as the Zn(II) ion is “invisible” to most spectroscopic techniques. The most common approaches to circumvent this inherent problem use other metal ions such as Co(II) (for UV/Vis spectroscopy) or Cd(II) (for NMR spectroscopy), which are relatively similar to Zn(II). Another approach uses coloured Zn(II) indicators. In the following the inventors describe results regarding the new mutant albumins obtained by ¹¹¹Cd NMR spectroscopy and titrations with Cu(II).

a) ¹¹¹Cd NMR of Mutant Albumins

¹¹¹Cd (or ¹¹³Cd; both nuclei can be used) NMR experiments are a relatively straightforward method to probe metal binding in a protein, provided the metal-loaded protein can be prepared with isotopically enriched Cd(II). The results of the present studies reveal interesting alterations in the metal binding properties of both mutants.

i) Asn99His Mutant

FIGS. 9 and 10 compare the 1D ¹¹¹Cd spectra of wild-type and Asn99Asp mutant albumins under identical conditions.

The Figures clearly show that, as in the case of wild-type rHA, two peaks can be observed in the 1D ¹¹¹Cd spectrum of the Asn99His mutant, suggesting that two equivalents of ¹¹¹Cd²⁺ are readily bound. Note that the line widths of the peaks in wild-type and mutant rHA spectra are comparable. The chemical shifts of the peaks are 122 (peak A) and 28 ppm (peak B) in the presence of 80 mM chloride. Compared to wild-type rHA (131 ppm and 27 ppm), this means that the metal binding site B is not affected by the mutation, whereas the Cd(II) ion in the mutant site A is a little more shielded than in the wild-type.

It might be predicted that substitution of an oxygen with a nitrogen donor would lead to deshielding, but the observed movement of peak A towards lower ppm values can be qualitatively understood if we assume that the N—Cd bond in the mutant is much shorter than the O—Cd bond in the wild-type. This is a reasonable assumption, because Asn is a very weak ligand, and the inventors have previously estimated that the O—Zn bond is around 2.15 Å long, compared to 1.95-2.00 Å for a Zn—N(His) bond. A similar trend is expected for Cd.

The inventors also probed the competition between Cd²⁺ and Zn²⁺ by adding Zn²⁺ to Cd₂rHA samples. Addition of Zn²⁺ clearly influences peak A, but even after addition of 3 equivalents, ¹¹¹Cd peak A is still present in the spectrum. In contrast, 1 mol equiv of Zn²⁺ is sufficient to completely obliterate peak A in wild-type rHA spectra, suggesting that Cd²⁺ has been displaced completely. The findings can be interpreted, to some extent, by considering the hard and soft acids and bases principle. Cd²⁺ is a “softer” metal ion than Zn²⁺, and nitrogen is a “softer” ligand than oxygen. Rendering the binding site “softer” will make Cd²⁺ binding more favourable than in wild-type rHA. Essentially, the experiments show that Asn99 contributes to the zinc site in wild-type rHA, and that the mutated site can indeed bind Cd²⁺ and Zn²⁺.

ii) Asn99Asp Mutant

FIG. 10 summarises the results on ¹¹¹Cd²⁺ binding studies on the Asn99Asp mutant rHA in comparison with wild-type rHA.

The occupation of Cd²⁺ site B again appears to be unaffected by the mutation (28 ppm, compared to 27 ppm in the wild-type spectra). It can be concluded that the mutation does not affect folding of this particular part of the protein, but site B is, as yet, unidentified.

Surprisingly, under the inventors' usual conditions for ¹¹¹Cd spectra (see FIG. 10, legend), no peak A was detected. Extending the chemical shift scale did not reveal any more peaks. To ensure that enough ¹¹¹Cd was available, 2 more equivalents of ¹¹¹Cd were added. In the spectrum recorded for this sample, there is a suggestion of two more peaks, but only at elevated temperature (310 K) two new, very broad resonances (with line-widths of almost 2000 Hz, compared to ca. 150-200 Hz for peak B) were detected with certainty. This suggests that chemical exchange phenomena influence the spectra. Despite the excess Cd(II) present, no precipitate was observed in the NMR tube, presumably due to the fact that Tris binds Cd²⁺ and thus solubilises it. The chemical shift for the Cd/Tris complex is 106 ppm (at 295 K), and this is also the value observed for peak C in FIG. 10. The peak A′ in the 310 K spectrum has a shift of 67 ppm, well in the range for ¹¹¹Cd sites with one nitrogen and between 3 and 5 oxygen donors (Coleman, J. A., Methods Enzymol. 227, 16-43 (1993); Öz, G. L., Pountney, D. L. & Armitage, I. M. Biochem. Cell Biol.—Biochim. Biol. Cell. 76, 223-234 (1998). The inventors hypothesise that the available ¹¹¹Cd is in intermediate (295 K) or slow (310 K) exchange between the mutated binding site A′ and Tris (or, alternatively, nonspecific sites on the protein).

b) Binding of Copper(II) Monitored by UV-Vis Spectroscopy

It has been shown previously (see also FIG. 6b) that addition of Cu²⁺ to wild-type rHA leads to the obliteration of the ¹¹¹Cd peak A, indicating that in vitro Cu²⁺ can also bind to this zinc site.

The inventors carried out titrations of Cu²⁺ into apo forms of wild-type and mutant albumins by UV-Vis spectroscopy, because such experiments provide quick qualitative information about metal binding, although a quantitative evaluation is not straightforward. The experiments shown in FIG. 11 reveal that Cu²⁺ binding to the Asn99 mutants differs from that to the wild-type. It is also clear that they do bind Cu²⁺ at a secondary site, as can be seen from the comparison with the His67Ala mutant rHA. The absorption profiles of the two mutants also differ from one another, implying the involvement of the mutated ligands.

Thus, the mutations have indeed affected the secondary Cu²⁺ site, which is known to be the primary Zn²⁺ site.

5. ¹H NMR of Mutant Albumins

The inventors have obtained 1D and 2D ¹H NMR spectra and FIG. 14 compares the aromatic region of 1D ¹H spectra of all mutants studied. FIG. 15 contains relevant portions of 2D TOCSY spectra for wild-type with and without zinc. Similar spectra for all mutants tested have been obtained (data not shown). All spectra are overall relatively similar to the wild-type spectrum. This indicates that none of the mutations has dramatic effects on the protein fold, at least not in the apo form. There are however subtle changes that can be interpreted.

In particular, peaks 1 and 3 in the wild-type NMR spectra are affected by any of the mutations considered (His67Ala, Asn99Asp, and Asn99His). It is therefore hypothesised that these can be assigned to residues His67 and His247.

Conclusions: Analysis of the 1D and 2D ¹H NMR spectra of wild-type and His67Ala, Asn99Asp, and Asn99His mutant rHA is consistent with the proposed binding site being formed by residues His67, Asn99, His247, and Asp249. Mutation of His67Ala affects two cross-peaks in the wild-type spectra, consistent with the idea that the mutated residue is in vicinity to another His (His247). Mutation of Asn99 also affects the same two wild-type cross-peaks, again suggesting that the three residues mentioned are indeed in close contact to each other. Cross-peaks 1 and A are assigned to His247 and cross-peaks 3 and B to His67.

6. ¹H NMR: Histidine Hε1 Resonances as Diagnostic Probes for Binding Events

After having assigned the two peaks corresponding to His67 and His247, it is important to explore whether there is an effect on these residues upon zinc binding.

a) Zinc Binding

The addition of 1 mol equivalent of Zn²⁺ to wild-type albumin has dramatic consequences on the dynamics of His residues as judged from 1D and 2D spectra.

Several peaks are heavily affected, whereas peaks 4, 6, 7, 8 and 11 remain unchanged. Peaks 1 and 3 are no longer observed, but also peak 5 has disappeared, and peaks 2 and 9/10 are reduced in intensity.

The fact that the peaks rather disappear than shift can be due to two effects of Zn²⁺ binding. Either the residue become more rigid upon zinc binding, which would lead to line-broadening, or zinc (and thus its ligands) exchanges between a free and a bound state, and this chemical exchange phenomenon also can lead to line-broadening. In any case, we have been able to establish that zinc binding can be monitored by ¹H NMR, and affects the residues His67 and His247, but also influences, directly or indirectly, other yet unidentified histidine residues.

Zinc Binding to Asn99Asp and Asn99His

Both 1 mM NMR samples containing 1 mol equiv of Zn(II) gave rise to precipitates after being kept at 310 K for about 30 min (the time it takes to acquire a 1D spectrum). No precipitate had been observed before the samples were introduced into the magnet, and after keeping the samples overnight at 279 K, the precipitate had dissolved again. The observed effect was more pronounced for the Asn99Asp mutant than for the Asn99His mutant. At present the inventors can only speculate that zinc has a pronounced effect on conformational dynamics, possibly related to inter-domain interactions. This idea is also consistent with the observations made with the wild-type. No precipitate was observed with the ¹¹¹Cd samples, although the Asn99Asp mutant sample also was subjected to 310 K for several hours.

b) Effect of Fatty Acid Binding to Wild-Type rHA

Albumin plays a vital role in the transport of otherwise insoluble long-chain fatty acids in blood plasma. Under normal conditions, 1-2 fatty acid molecules are bound to albumin, but during exercise, this number can rise to 4. (Peters, T., Jr. All About Albumin: Biochemistry, Genetics, and Medical Applications. Academic Press, New Your (1995)). The maximum number observed in vivo is 6, although X-ray structures of albumin show between 5 (Curry, S., M,andelkow, H., Brick P. & Franks, N. Nat. Strict. Biol. 5, 827-835 (1998) and 10 (Bhattacharya, A. A., Grüne, T. & Curry, S. J. Mol. Biol. 303, 721-732 (2000) fatty acid bindings sites. Binding of fatty acids to albumin has been extensively studied in the past, using a variety of techniques including ¹³C (Hamilton, J. A., Era, S., Bhamidipati, S. P. & Reed, R. G. Proc. Natl. Acd. Sci. U.S.A. 88, 2051-4 (1991) and ¹H NMR (Oida, T. J. Biochem. (Japan) 100, 1533-42 (1986).

It can be seen immediately in FIG. 17 that numerous His Hε1 peaks are substantially affected by fatty acid binding. This gives a handle on monitoring the effects of interactive zinc and fatty acid binding.

7. ¹¹¹Cd NMR as a Probe for Interactive Metal/Fatty Acid Binding

Initial ¹¹¹Cd NMR spectroscopy experiments with octanoate-saturated rHA samples had revealed that peak A is absent in the spectra of such samples.

Starting with an exhaustively dialysed sample (the inventors have also established that there is no discernible difference between the 1D ¹¹¹Cd NMR spectra of such dialysed samples or Chen-defatted preparations.) containing 2 mol equivs of ¹¹¹Cd²⁺, we added equivalents of potassium octanoate. Figure X shows the results of the titration study.

The most striking finding is that peak A initially diminishes, but re-develops after several hours. There seems to be a slow equilibrium, which leads to the re-distribution of fatty acid and metal ions. Initial binding of fatty acid to site F2 appears to be relatively fast, as the decrease in peak intensity can be observed directly after mixing the sample (at least for the addition of 2 or 3 equivalents; the dynamics appear to slow down in the subsequent additions). As peak A re-emerges, it can be speculated that the fatty acid molecule subsequently is relocated to a thermodynamically more favoured binding site. Dissociation of fatty acid from site F2 is expected to allow the re-formation of the metal-binding site, and available Cd²⁺ can be bound again. This procedure can be repeated up to 4 equivalents, then all fatty acid binding sites with higher thermodynamic stability than site F2 appear to be saturated. In the final spectrum of a sample containing 5 equivalents of octanoate, peak A is not present any more.

An important result of this study is the conclusion that binding of fatty acids does not only prevent metal binding, but that binding of metal and fatty acids is an interactive process, and the binding of fatty acid anions appears to lead ultimately to the dissociation of bound metal ion.

8. Cell Experiments

Albumin has been found to protect liver tissue against ischemia- and hypoxia-induced hepatic injury, and the effect has been attributed to albumin's metal binding capacity (Strubelt, O., Younes, M., Li, Y. Pharmacology and Toxicology 75, 280-284 (1994). The inventors have developed in vitro experiments in order to explore the effects of zinc, recombinant human serum albumin, and mutant albumins on hepatocyte cell cultures.

The human hepatocytes cell line used was WRL-68. The cells were grown in Dulbecco's minimal medium. For investigating the effects of rHA of the His67Ala mutant albumin, 600 μM rHA or His67Ala mutant were added to the medium. The effects of Zn(II) were explored by adding 60, 300, and 600 μM ZnCl₂ to the medium.

These initial experiments were evaluated by counting cells and assessing cell viability using Trypan Blue. They suggested a distinct negative effect of elevated levels of zinc on cell viability and adhesion, which can be rescued by addition of wild-type albumin, but also that the His67Ala mutant is cytotoxic, and inhibits cell adhesion.

Subsequently, in order to generate cell layers prior to albumin treatment, human WRL-68 hepatocytes were cultivated for 18 h before being incubated for 48 h with Dulbecco's minimal eagles medium supplemented with different doses (60, 300, and 600 μM) of Zn in the presence or absence of wild-type or mutant albumins (600 μM). Otherwise, growth conditions (37 C, 5% CO₂) were identical to the previous experiments. The inventors have also extended the studies to the two new mutants, Asn99Asp and Asn99His.

The graphs in FIG. 18 summarise the effects of the combined treatment of human hepatocytes with zinc and different mutant albumins. The hepatocytes were grown in layers in 12-well plates, and cell counts and viability were determined both in layers and medium. All experiments have been carried out in triplicate, the error bars correspond to the standard deviation between individual runs.

The following conclusions can be drawn from the results presented in FIGS. 18a-d.

a) It was confirmed that elevated levels of Zn²⁺ lead to cell death and loss of adhesion.

b) Wild-type rHA is very well tolerated by the cells; there is no significant difference in growth or adhesion between the cell counts in the controls and those containing wild-type rHA.

c) The adverse effects of Zn²⁺ are reversed by 600 μM wild-type rHA.

d) The His67Ala mutant, without added Zn²⁺, has a dramatic effect on cell adhesion, leading to the majority of cells floating in the medium, although cell viability does not seem to be impaired in either layer or medium.

e) Addition of Zn²⁺ surprisingly appears to reverse the negative effect of His67Ala on cell adhesion, without exerting the same damaging effects if given alone.

f) Treatment with the Asn99Asp mutant albumin leads to increased cell growth (ca.+20%), irrespective of the amount of added Zn²⁺.

g) Treatment with the Asn99His mutant rHA leads to a most dramatic loss of adhesion. Contrary to the His67Ala mutant rHA, addition of Zn²⁺ has no beneficial effects. Cell viability does not seem to be affected.

In summary, the inventors have shown that mutations to the zinc site ligands has far-reaching consequences, both for the physicochemical properties of the protein, but also for its effects on living cells. Although the reasons for the various effects observed remain to be established, the inventors speculate, without wishing to be bound by theory, that conformational dynamics, domain/domain interactions, protein/protein interactions, and maybe protein/membrane interactions are responsible for most of the present observations.

As a result of these studies it is possible to prepare novel mutant albumins with decreased or enhanced affinities for metal ions such as Zn²⁺ by mutation of residues around the locus of the metal site. These include mutation of a side-chain, which can bind metals to one which cannot (or only weakly) bind and is likely to give rise to decreased metal affinity.

TABLE 1

| | | |
|---|---|---|
| HUMAN MACQUE CANINE FELINE BOVINE SHEEP PIG RABBIT RAT | 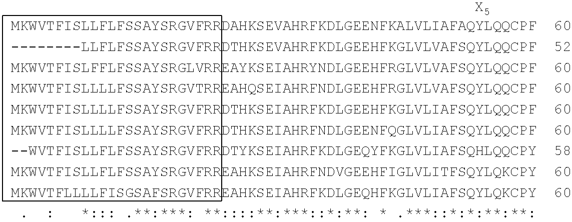 | |
| | X₁ | |
| HUMAN | EDHVKLVNEVTEFAKTCVADESAENCDKSLHTLFGDKLCTVATLRETYGEMADCCAKQEP 120 | |
| MACAQUE | EEHVKLVNEVTEFAKTCVADESAENCDKSLHTLFGDKLCTVATLRETYGEMADCCAKQEP 112 | |
| CANINE | EDHVKLAKEVTEFAKACAAEESGANCDKSLHTLFGDKLCTVASLRDKYGDMADCCEKQEP 120 | |
| FELINE | EDHVKLVNEVTEFAKGCVADQSAANCEKSLHELLGDKLCTVASLRDKYGEMADCCEKKEP 120 | |
| BOVINE | DEHVKLVNELTEFAKTCVADESHAGCEKSLHTLFGDELCKVASLRETYGDMADCCEKQEP 120 | |
| SHEEP | DEHVKLVKELTEFAKTCVADESHAGCDKSLHTLFGDELCKVATLRETYGDMADCCEKQEP 120 | |
| PIG | EEHVKLVREVTEFAKTCVADESAENCDKSIHTLFGDKLCAIPSLREHYGDLADCCEKEEP 118 | |
| RABBIT | EEHAKLVKEVTDLAKACVADESAANCDKSLHDIFGDKICALPSLRDTYGDVADCCEKKEP 120 | |
| RAT | EEHIKLVQEVTDFAKTCVADENAENCDKSIHTLFGDKLCAIPKLRDNYGELADCCAKQEP 120 | |
| | ::* **..*:*::** *.*::. .*:**:* ::**::* :..**: **::**** *:** | |
| | X₂X₈ X₆ X₉ | |
| HUMAN | ERNECFLQHKDDNPNLPRLVRPEVDVMCTAFHDNEETFLKKYLYEIARRHPYFYAPELLF 180 | |
| MACAQUE | ERNECFLQHKDDNPNLPPLVRPEVDVMCTAFHDNEATFLKKYLYEVARRHPYFYAPELLF 172 | |
| CANINE | DRNECFLAHKDDNPGFPPLVAPEPDALCAAFQDNEQLFLGKYLYEIARRHPYFYAPELLY 180 | |
| FELINE | ERNECFLQHKDDNPGFGQLVTPEADAMCTAFHENEQRFLGKYLYEIARRHPYFYAPELLY 180 | |
| BOVINE | ERNECFLSHKDDSPDLPKLK-PDPNTLCDEFKADEKKFWGKYLYEIARRHPYFYAPELLY 179 | |
| SHEEP | ERNECFLNHKDDSPDLPKLK-PEPDTLCAEFKADEKKFWGKYLYEVARRHPYFYAPELLY 179 | |
| PIG | ERNECFLQHKNDNPDIPKLK-PDPVALCADFQEDEQKFWGKYLYEIARRHPYFYAPELLY 177 | |
| RABBIT | ERNECFLHHKDDKPDLPPFARPEADVLCKAFHDDEKAFFGHYLYEVARRHPYFYAPELLY 180 | |
| RAT | ERNECFLQHKDDNPNLPPFQRPEAEAMCTSFQENPTSFLGHYLHEVARRHPYFYAPELLY 180 | |
| | :****** **:*.*.: : *: .:* *: : * :**:*:*************: | |
| HUMAN | FAKRYKAAFTECCQAADKAACLLPKLDELRDEGKASSAKQRLKCASLQKFGERAFKAWAV 240 | |
| MACAQUE | FAARYKAAFAECCQAADKAACLLPKLDELRDEGKASSAKQRLKCASLQKFGDRAFKAWAV 232 | |
| CANINE | YAQQYKGVFAECCQAADKAACLGPKIEALREKVLLSSAKERFKCASLQKFGDRAFKAWSV 240 | |
| FELINE | YAEEYKGVFTECCEAADKAACLTPKVDALREKVLASSAKERLKCASLQKFGERAFKAWSV 240 | |
| BOVINE | YANKYNGVFQECCQAEDKGACLLPKIETMREKVLASSARQRLRCASIQKFGERALKAWSV 239 | |
| SHEEP | YANKYNGVFQECCQAEDKGACLLPKIDAMREKVLASSARQRLRCASIQKFGERALKAWSV 239 | |
| PIG | YAIIYKDVFSECCQAADKAACLLPKIEHLREKVLTSAAKQRLKCASIQKFGERAFKAWSL 237 | |
| RABBIT | YAQKYKAILTECCEAADKGACLTPKLDALEGKSLISAAQERLRCASIQKFGDRAYKAWAL 240 | |
| RAT | YAEKYNEVLTQCCTESDKAACLTPKLDAVKEKALVAAVRQRMKCSSMQRFGERAFKAWAV 240 | |
| | :* *: : :** **.*** **:: :. : ::.::*::*:*:*:**:** ***:: | |
| | X₁₀ X₃X₇X₄ | |
| HUMAN | ARLSQRFPKAEFAEVSKLVTDLTKVHTECCHGDLLECADDRADLAKYICENQDSISSKLK 300 | |
| MACAQUE | ARLSQKFPKAEFAEVSKLVTDLTKCHTECCHGDLLECADDRADKALYMCENQDSISSKLK 292 | |
| CANINE | ARLSQRFPKADFAEISKVVTDLTKVHKECCHGDLLECADDRADLAKYMCENQDSISTKLK 300 | |
| FELINE | ARLSQKFPKAEFAEISKLVTDLAKIHKECCHGDLLECADDRADLAKYICENQDSISTKLK 300 | |
| BOVINE | ARLSQKFPKAEFVEVTKLVTDLTKVHKECCHGDLLECADDRADLAKYICDNQDTISSKLK 299 | |
| SHEEP | ARLSQKFPKADFTDVTKIVTDLTKVHKECCHGDLLECADDRADLAKYICDHQDALSSKLK 299 | |
| PIG | ARLSQRFPKADFTEISKIVTDLAKVHKECCHGDLLECADDRADLAKYICENQDTISTKLK 297 | |
| RABBIT | VRLSQRFPKADFTDISKIVTDLTKVHKECCHGDLLECADDRADLAKYMCEHQETISSHLK 300 | |
| RAT | ARMSQRFPNAEFAEITKLATDVTKINKECCHGDLLECADDRAELAKYMCENQATISSKLQ 300 | |
| | .*:**:**:*:*.:::*:.**::*::.***************:****:*::* ::*::*: | |
| | X₁₁ | |
| HUMAN | ECCEKPLLEKSHCIAEVENDEMPADLPSLAADFVESKDVCKYAEAKDVFLGMFLYEYAR 360 | |
| MACAQUE | ECCEKPLLEKSHCIAEVENDEMPADLPSLAADFVESKDVCKNYAEAKDVFLGMFLYEYAR 360 | |
| CANINE | ECCDKPVLEKSQCLAEVERDELPGDLPSLAADFVEDKEVCKNYQEAKDVFLGTFLYEYSR 360 | |
| FELINE | ECCGKPVLEKSHCISEVERDELPADLPPLAVDFVEDKEVCKNYQEAKDVFLGTFLYEYSR 360 | |
| BOVINE | ECCDKPLLEKSHCIAEVEKDAIPENLPPLTADFAEDKDVCKNYQEAKDAFLGSFLYEYSR 359 | |
| SHEEP | ECCDKPVLEKSHCIAEVDKDAVPENLPPLTADFAEDKEVCKNYQEAKDVFLGSFLYEYSR 359 | |
| PIG | ECCDKPLLEKSHCIAEAKRDELPADLNPLEHDFVEDKEVCKNYKEAKDVFLGTFLYEYSR 357 | |
| RABBIT | ACCDKPILEKAHCIYGLHNDEDTAGLPAVAEEFVEDKDVCKNYEEAKDLFLGKFLYEYSR 360 | |
| RAT | ACCDKPVLQKSQCLAETEHDNIPADLPSIAADFVEDKEVCKNYAEAKDVFLGTFLYEYSR 360 | |
| | ** **:*:*::*: ..* . .* .: ::.*.*:***** **** *** *****:* | |
| HUMAN | RHPDYSVVLLLRLAKTYETTLEKCCAAADPHECYAKVFDEFKPLVEEPQNLIKQNCELFE 420 | |
| MACAQUE | RHPDYSVMLLLRLAKAYEATLEKCCAAADPHECYAKVFDEFQPLVEEPQNLVKQNCELFE 412 | |
| CANINE | RHPEYSVSLLLRLAKEYEATLEKCCATDDPPTCYAKVLDEFKPLVDEPQNLVKTNCELFE 420 | |
| FELINE | RHPEYSVSLLLRLAKEYEATLEKCCATDDPPACYAHVFDEFKPLVEEPHNLVKTNCELFE 420 | |
| BOVINE | RHPEYAVSVLLRLAKEYEATLEECCAKDDPHACYSTVFDKLKHLVDEPQNLIKQNCDQFE 419 | |
| SHEEP | RHPEYAVSVLLRLAKEYEATLEDCCAKEDPHACYATVFDKLKHLVDEPQNLIKKNCELFE 419 | |
| PIG | RHPDYSVSLLLRIAKIYEATLEDCCAKEDPPACYATVFDKFQPLVDEPKNLIKQNCELFE 417 | |
| RABBIT | RHPDYSVVLLLRLGKAYEATLKKCCATDDPHACYAKVLDEFQPLVDEPKNLVKQNCELYE 420 | |
| RAT | RHPDYSVSLLLRLAKKYEATLEKCCAEGDPPACYGTVLAEFQPLVEEPKNLVKTNCELYE 420 | |
| | ***:*:* :***:.* **:**:.*** ** **. *: ::: **:**:**:* **: :* | |
| HUMAN | QLGEYKFQNALLVRYTKKVPQVSTPTLVEVSRNLGKVGSKCCKHPEAKRMPCAEDYLSVV 480 | |
| MACAQUE | QLGEYKFQNALLVRYTKKVPQVSTPTLVEVSRNLGKVGAKCCKLPEAKRMPCAEDYLSVV 472 | |
| CANINE | KLGEYGFQNALLVRYTKKAPQVSTPTLVEVSRKLGKVGTKCCKKPESERMSCADDFLSVV 480 | |
| FELINE | KLGEYGFQNALLVRYTKKVPQVSTPTLVEVSRSLGKVGSKCCTHPEAERLSCAEDYLSVV 480 | |
| BOVINE | KLGEYGFQNALIVRYTRKVPQVSTPTLVEVSRSLGKVGTRCCTKPESERMPCTEDYLSLI 479 | |
| SHEEP | KHGEYGFQNALIVRYTRKAPQVSTPTLVEISRSLGKVGTKCCAKPESERMPCTEDYLSLI 479 | |
| PIG | KLGEYGFQNALIVRYTKKVPQVSTPTLVEVARKLGLVGSRCCKRPEEERLSCAEDYLSLV 477 | |
| RABBIT | QLGDYNFQNALLVRYTKKVPQVSTPTLVEISRSLGKVGSKCCKHPEAERLPCVEDYLSVV 480 | |
| RAT | KLGEYGFQNAVLVRYTQKAPQVSTPTLVEAARNLGRVGTKCCTLPEAQRLPCVEDYLSAI 480 | |
| | : *:* ****::****:*.********** :*.** **::** ** :*:.*.:*:** : | |
| HUMAN | LNQLCVLHEKTPVSDRVTKCCTESLVNRRPCFSALEVDETYVPKEFNAETFTFHADICTL 540 | |
| MACAQUE | LNRLCVLHEKTPVSEKVTKCCTESLVNRRPCFSALELDEAYVPKAFNAETFTFHADMCTL 532 | |
| CANINE | LNRLCVLHEKTPVSERVTKCCSESLVNRRPCFSGLEVDETYVPKEFNAETFTFHADLCTL 540 | |
| FELINE | LNRLCVLHEKTPVSERVTKCCTESLVNRRPCFSALQVDETYVPKEFSAETFTFHADLCTL 540 | |
| BOVINE | LNRLCVLHEKTPVSEKVTKCCTESLVNRRPCFSALTPDETYVPKAFDEKLFTFHADICTL 539 | |
| SHEEP | LNRLCVLHEKTPVSEKVTKCCTESLVNRRPCFSDLTLDETYVPKPFDEKFFTFHADICTL 539 | |
| PIG | LNRLCVLHEKTPVSEKVTKCCTESLVNRRPCFSALTPDETYKPKEFVEGTFTFHADLCTL 537 | |
| RABBIT | LNRLCVLHEKTPVSEKVTKCCSESLSNRRPCFSALGPDETYVPKEFNAETFTFHADICTL 540 | |
| RAT | LNRLCVLHEKTPVSEKVTKCCSGSLVERRPCFSALTVDETYVPKEFKAETFTFHSDICTL 540 | |
| | **:***********::*****: ** :****** * **:* ** * ****:*:*** | |
| HUMAN | SEKERQIKKQTALVELVKHKPKATKEQLKAVMDDFAAFVEKCCKADDKETCFAEEGKKLV 600 | |
| MACAQUE | SEKEKQVKKQTALVELVKHKPKATKEQLKGVMDNFAAFVEKCCKADDKEACFAEEGPKFV 592 | |
| CANINE | PEAEKQVKKQTALVELLKHKPKATDEQLKTVMGDFGAFVEKCCAAENKEGCFSEEGPKLV 600 | |
| FELINE | PEAEKQIKKQSALVELLKHKPKATEEQLKTVMGDFGSFVDKCCAAEDKEACFAEEGPKLV 600 | |
| BOVINE | PDTEKQIKKQTALVELLKHKPKATEEQLKTVMENFVAFVDKCCAADDKEACFAVEGPKLV 599 | |
| SHEEP | PDTEKQIKKQTALVELLKHKPKATDEQLKTVMENFVAFVDKCCAADDKEGCFVLEGPKLV | 599 |
| PIG | PEDEKQIKKQTALVELLKHKPHATEEQLRTVLGNFAAFVQKCCAAPDHEACFAVEGPKFV 597 | |
| RABBIT | PETERKIKKQTALVELVKHKPHATNDQLKTVVGEFTALLDKCCSAEDKEACFAVEGPKLV 600 | |
| RAT | PDKEKQIKKQTALAELVKHKPKATEDQLKTVMGDFAQFVDKCCKAADKDNCFATEGPNLV 600 | |
| | .: *:::***:**.**:****:**.:**: *: :* :::*** * ::: ** ** ::* | |
| HUMAN | AASQAALGL 609 | |
| MACAQUE | AASQAALA- 600 | |
| CANINE | AAAQAALV- 608 | |
| FELINE | AAAQAALA- 608 | |
| BOVINE | VSTQTALA- 607 | |
| SHEEP | ASTQAALA- 607 | |
| PIG | IEIRGILA- 605 | |
| RABBIT | ESSKATLG- 608 | |
| RAT | ARSKEALA- 608 | |
| | : * | |

Comparison of amino acid sequence between mammalian albumins. Residues, which may be mutated are highlighted. Amino acids before the N terminal amino acid (residue number 1), in the boxed area, are part of the pre-albumin sequence and are cleaved following translation to give albumin itself. Accession numbers of the sequences are Human, P02768; Macaque, M90463; Canine, CAB64867; Feline, P49064; Bovine, P02769; Sheep, P14639; Pig, ABPGS; Rabbit, P49065 and Rat, P02770.

1. An isolated mutant human serum albumin substantially comprising the amino acid sequence:
(SEQ ID NO.:1)
DAHKSEVAHRFKDLGEENFKALVLIAFAQX₅LQQCPFEDHVKLVNEVTEF
AKTCVADESAENCDKSLX₁TLFGDKLCTVATLRETYGEMADCCAKQEPER
X₂X₈CFX₆QHKDDNPNLPRLVRPEVDVMCTAFHDNEETFLKKYLYEIARR
X₉PYFYAPELLFFAKRYKAAFTECCQAADKAACLLPKLDELRDEGKASSA
KQRLKCASLQKFGERAFKAWAVARLSQRFPKAEFAEVSKLVTDLTKVX₁₀
TECCX₃X₇X₄LLECADDRADLAKYICENQDSISSKLKECCEKPLLEKS
X₁₁CIAEVENDEMPADLPSLAADFVESKDVCKNYAEAKDVFLGMFLYEYA
RRHPDYSVVLLLRLAKTYETTLEKCCAAADPHECYAKVFDEFKPLVEEPQ
NLIKQNCELFEQLGEYKFQNALLVRYTKKVPQVSTPTLVEVSRNLGKVGS
KCCKHPEAKRMPCAEDYLSVVLNQLCVLHEKTPVSDRVTKCCTESLVNRR
PCFSALEVDETYVPKEFNAETFTFHADICTLSEKERQIKKQTALVELVKH
KPKATKEQLKAVMDDFAAFVEKCCKADDKETCFAEEGKKLVAASQAALGL
wherein X₁, is other than H; X₂ is other than N, X₃ is other than H, X₄ is other than D; X₅ is other than Y; X₆ is other than L; X₇ is other than G, X₈ is other than E, X₉ is other than H, X₁₀ is other than H, and X₁₁ is other than H, such that said mutant displays an altered metal binding affinity or one or more physiological characteristics with respect to native human serum albumin.

2. The mutant according to claim 1 wherein said one or more physiological characteristics are a change in cell adhesion to a substrate, percentage viability of cell, or cell growth of cells in culture.

3. An isolated mutant mammalian serum albumin substantially comprising one of the sequences as shown in Table 1 wherein at least one of the residues denoted by Xₙ is mutated such that said mutant serum albumin displays an altered metal binding affinity or one or more physiological characteristics with respect to the native sequence from which the mutant is derived.

4. An isolated mutant serum albumin according to claims 1 or 3 which is at least 90% identical with the native sequence from which the mutant is derived.

5. The mutant serum albumin according to claims 1 or 3 which is substantially similar in terms of general overall folding with respect to the native serum albumin from which it is derived.

6. The mutant serum albumin according to claims 1 or 3 wherein the altered metal binding affinity is a decrease or increase in metal binding affinity.

7. The mutant according to claims 1 or 3 wherein the metal is zinc.

8. The mutant according to claims 1 or 3 comprising at least one of the following mutations:
X₁=>A, F, G, I, K, L, N, P, Q, R, S, T, V, W, Y, C, D, E
X₂=>A, F, G, I, K, L, P, Q, R, S, T, V, W, Y, C, D E, H
X₃=>A, F, G, I, K, L, N, P, Q, R, S, T, V, W, Y, C, D, E
X₄=>A, F, G, I, K, L, N, P, Q, R, S, T, V, W, Y, C, E, H
X₅=>C, D, E, H
X₆=>C, D, E, H
X₇=>C, D, E, H
X₈=>A, C, F, G, H, I, K, L, N, P, Q, R, S, T, V, W, Y
X₉=>A, D, E, F, G, I, K, L, N, P, Q, R, S, T, V, W, Y
X₁₀=>A, D, E, F, G, I, K, L, N, P, Q, R, S, T, V, W, Y
X₁₁=>A, D, E, F, G, I, K, L, N, P, Q, R, S, T, V, W, Y

9. The mutant according to claims 1 or 3 comprising at least one mutation at X₁, X₂, X₃ or X₄.

10. A mutant human serum albumin comprising the mutation Asn 99His, Asn99Asp or His67Ala.

11. A nucleic acid sequence capable of encoding a mutant serum albumin according to claims 1, 3 or 10.

12. An expression cassette comprising a promoter operably linked to a nucleic acid sequence according to claim 11.

13. A pharmaceutical composition comprising a mutant serum albumin, a nucleic acid sequence or an expression cassette according to claims 1 or 3 and a pharmaceutically acceptable carrier therefore.

14. A cell culture medium comprising a mutant serum albumin, a nucleic acid sequence or an expression cassette according to claims 1, 3 or 10.

15. (canceled)

16. A method of altering growth characteristics of cells in cell culture comprising the step of culturing cells in cell culture in the presence of a mutant serum albumin according to claims 1 or 3.

17. A method of obtaining a mutant serum albumin which displays an altered metal binding affinity or one or more physiological characteristics with respect to a native albumin from which the mutant has been derived, comprising the steps of:
a) providing a nucleic acid sequence encoding a nucleic albumin polypeptide;
b) conducting a mutagenesis reaction on said nucleic acid in order to alter said nucleic acid whereby said altered nucleic acid sequence encodes a mutant albumin polypeptide comprising at least one mutation with respect to said native albumin;
c) expressing said mutant albumin polypeptide and detecting whether or not said mutant albumin displays an altered metal binding or one or more physiological characteristics.

18. The method according to claim 17 wherein the mutant albumin comprises at least one mutation to residues X₁-X₁₁ as shown in Table 1.

19. The mutant according to claim 1 wherein the mutant displays an altered metal binding affinity and one or more physiological characteristic with respect to native human serum albumin.

20. The mutant according to claim 1 wherein said one or more physiological characteristics are a change in cell adhesion to a substrate, percentage viability of cell, and cell growth of cells in culture.

21. A method of obtaining a mutant serum albumin which displays an altered metal binding affinity and one or more physiological characteristics with respect to a native albumin from which the mutant has been derived, comprising the steps of:
a) providing a nucleic acid sequence encoding a nucleic albumin polypeptide;
b) conducting a mutagenesis reaction on said nucleic acid in order to alter said nucleic acid whereby said altered nucleic acid sequence encodes a mutant albumin polypeptide comprising at least one mutation with respect to said native albumin;
c) expressing said mutant albumin polypeptide and detecting whether or not said mutant albumin displays an altered metal binding and said one or more physiological characteristics.
